(19) United States
(12) Patent Application Publication
(10) Pub. No.: US 2018/0271924 A1
(43) Pub. Date: Sep. 27, 2018

(54) COMPOUND AND METHOD FOR REDUCING INFLAMMATION, PAIN, ALLERGY, FLU AND COLD SYMPTOMS

(71) Applicant: Alexander Kariman, Rockville, MD (US)

(72) Inventor: Alexander Kariman, Rockville, MD (US)

(21) Appl. No.: 15/992,204

(22) Filed: May 30, 2018

(51) Int. Cl. A61K 36/24; A61K 9/00; A61P 11/14; A61P 11/10; A61P 11/08; A61P 31/10; A61P 3/04; A61P 3/10; A61P 1/04; A61P 29/00

(52) U.S. Cl. CPC A61K 36/24; A61K 9/0014; A61K 9/0053; A61P 11/14; A61P 11/10; A61P 29/00; A61P 31/10; A61P 3/04; A61P 3/10; A61P 1/04; A61P 11/08

(57) ABSTRACT

The present invention relates to pharmaceutical and nutraceutical compounds and methods for reducing inflammation, blood sugar, gastric acid, symptoms of allergy, common cold and flu, as well as treating infections and pain associated with trauma, medical procedure, and diseases and disorders in subjects in need thereof. The compounds and methods proposed by this invention are related to essentially a mixture of naturally accruing and/or synthetic substances in certain ratios, administration protocols, and delivery systems that amplify their medicinal qualities and reduce side effects, making their clinical application feasible.

### FIELD OF THE INVENTION

The present invention relates to pharmaceutical and nutraceutical compounds and methods for reducing inflammation, blood sugar levels, gastric acid, symptoms of allergy, common cold and flu, as well as treating infections and pain associated with trauma, medical procedure, and diseases and disorders in subjects in need thereof.

### BACKGROUND OF THE INVENTION

The compounds and methods proposed herein are related to essentially a mixture of naturally accruing and synthetic substances to produce novel compounds that exhibit strong medicinal qualities. A plurality of medicinal and dietary concoctions can be manufactured from the disclosed compounds with multiple pharmacological effects and having different methods of administration. The active ingredients of said compounds can be derived from Cannabis sativa, Picralima nitida, Mitragyna speciose, Vinca major, and several Psychotria species plants, or can be chemically synthesized.

The utility of this invention comes from the prescribing protocols and formulations designed to amplify the therapeutic value of the ingredients and reduce side effects, making their use in medicine practical. For instance, the effect of cannabinoid (CB)-based medicine, such as Dronabinol, is dose dependent. Likewise, the effect of Mitragyna speciose extract, which is a stimulant and sedative depending on the dose. The most frequently reported side effects of cannabinoids, which is a limiting factor in their application, are the mental slowness, impaired reaction times, and sometimes accentuation of anxiety (No authors listed, 2011). However, the lower doses in certain treatments, such as Multiple Sclerosis (MS)-related spasticity, may result in diminished efficacy. Likewise, a larger dose of mitragynine, required for reliable analgesia causes the unwanted sedation, constipation, and other side effects. Furthermore, the Psychotria umbellate plant alkaloids may cause dissociative psychotropic effects at larger doses. Therefore, in addition to the treatment protocols disclosed herein, the proposed formulations are designed to increase the treatment safety and therapeutic benefits by exploiting the ratios and the synergetic effects of the active constituents to achieve the desired therapeutic outcomes. An overview of the plants, their constituents, safety and efficacy are provided below.

Picralima nitida (syn.: Tabernaemontana nitida, Picralima klaineana, Picralima macrocarpa, Tabernaemontana macrocarpa) is the only species of the genus Picralima and it is related to Hunteria and Pleiocarpa. Picralima nitida is commonly called Picralima, Akuamma or Pile plant, it belongs to the Hunterieae tribe of the Apocynaceae family. The plant is widely distributed in high deciduous forest of West-Central Africa from Ivory Coast to West Cameroons and extending across the Congo basin and Uganda (Erharuyi, Falodun, & Langer, 2014). Picralima nitida is an understory tree which reaches up to 35 m in height, crown dense, trunk is 5-60 cm in diameter, cylindrical, the wood is pale yellow, hard, elastic, fine-grained and taking a high polish (Erharuyi, Falodun, & Langer, 2014). Picralima nitida bears white flowers (about 3 cm long) with ovoid fruits which at maturity are yellowish in color (Erharuyi, Falodun, & Langer, 2014). The leaves are broad (3-10 cm) and oblong (6-20 cm long) with tough tiny lateral nerves of about 14 to 24 pairs (Erharuyi, Falodun, & Langer, 2014).

Picralima nitida has many applications in West Africa's folk medicine. Various parts of the plant, leaves, seeds, stem bark and roots, are used by herbalists for the treatment of fever, hypertension, jaundice, gastro-intestinal disorders and for treatment of malaria (Erharuyi, Falodun, & Langer, 2014). The extract from different parts of the plant have been found to exhibit a broad range of pharmacological activities which lends credence to its ethnomedicinal uses (Erharuyi, Falodun, & Langer, 2014). The phytochemicals isolated from various parts of the plant are structurally related alkaloids as well as flavonoids, terpenoids, saponins, polyphenols, tannins, steroids and various glycosides. Indole alkaloids isolated from the seeds of Picralima nitida include: akuammine, akuammidine, akuammicine, akuammigine, pseudoakuammigine and others. Some of these alkaloids are central nervous system stimulants and depressants that can act upon a variety of neurotransmitter systems within the human brain. The extract from different parts of the plant have been found to exhibit a number of pharmacological activities which lends credence to its ethnomedicinal uses (Erharuyi, Falodun, & Langer, 2014). Vinca major (syn.: Bigleaf periwinkle, Large periwinkle, Greater periwinkle and Blue periwinkle) is a species of flowering plant in the family of Apocynaceae, native to the western Mediterranean. Growing. It grows 25 cm tall and spreads indefinitely. It is an evergreen perennial plant, frequently used in cultivation as groundcover. Vinca major is a trailing vine, spreading along the ground and rooting along the stems to form dense masses of groundcover individually 2-5 m across and scrambling up to 50-70 cm high. The leaves are opposite, nearly orbicular at the base of the stems and lanceolate at the apex, 3-9 cm long and 2-6 cm broad, glossy dark green with a leathery texture and an entire but distinctly ciliate margin, and a hairy petiole 1-2 cm long. The flowers are hermaphrodite, axillary and solitary, violet-purple, 3-5 cm diameter, with a five-lobed corolla. The calyx surrounding the base of the flower is 10-17 mm long with hairy margins. The flowering period extends from early spring to autumn. This species is found in southern Europe and northern Africa, from Spain and southern France east to the western Balkans, and also in northeastern Turkey and the western Caucasus. It prefers moist undergrowth, woodlands, hedgerows and banks along the rivers at an altitude of 0-800 meters above sea level. It grows well in full sun and in deep shade.

Cannabis sativa (marijuana), that is proposed in some embodiments of this invention, is an annual herbaceous flowering plant indigenous to eastern Asia but now of cosmopolitan distribution due to cultivation. It is placed in the Cannabis genus classification, which belongs to a small but diverse family, the Cannabaceae. CB components of marijuana are known to exert behavioral and psychotropic effects but also to possess therapeutic properties including analgesia, ocular hypotension, and antiemesis. CBs-based medications are now being used for treatment of a wide range of medical conditions, including neuropathic pain, pain related to cancer and trauma, spasticity associated with MS, fibromyalgia, and others.

The Cannabis sativa plant and its products consist of many chemicals. Some of the 483+identified compounds are unique to Cannabis. So far, according to Brenneisen (2007), 66 cannabinoids have been identified, and they are divided into 10 subclasses: 1) Cannabigerol class: cannabigerolic acid (CBGA)—antibiotic; cannabigerolic acid monomethylether (CBGAM); cannabigerol (CBG)—antibiotic, antifungal, anti-inflammatory, relaxant (possibly inhibits the uptake of GABA); cannabigerol monomethylether (CBGM); cannabigerovarinic acid (CBGVA); cannabigerovarin (CBGV); 2) Cannabichromene class: cannabichromenic acid (CBCA); cannabichromene (CBC)—anti-inflammatory, antibiotic, antifungal, analgesic; cannabichromevarinic acid (CBCVA); cannabichromevarin (CBCV); 3) Cannabidiol class: cannabidiolic acid (CBDA)—antibiotic; cannabidiol (CBD)—anxiolytic, antipsychotic, analgesic, anti-inflammatory, antioxidant, antispasmodic; cannabidiol monomethylether (CBDM); cannabidiol-C4 (CBD-C4); cannabidivarinic acid (CBDVA); cannabidivarin (CBDV); cannabidiorcol (CBD-C1); 4) Delta-9-tetrahydrocannabinol class: delta-9-tetrahydrocannabinolic acid A (THCA-A), delta-9-tetrahydrocannabinolic acid B (THCA-B), delta-9-tetrahydrocannabinol(THC)—euphoric analgesic, anti-inflammatory, antioxidant, antiemetic; delta-9-tetrahydrocannabinolic acid-C4 (THCA-C4), delta-9-tetrahydrocannabinol-C4(THC-C4); delta-9-tetrahydrocannab ivarinic acid (THCVA); delta-9-tetrahydrocannab ivarin (THCV)—analgesic, euphoriant; delta-9-tetrahydrocannab iorcolic acid (THCA-C1); delta-9-tetrahydrocannabiorcol (THC-C1); delta-7-cis-iso-tetrahydrocannabivarin; 5) Delta-8-tetrahydrocannabinol class: delta-8-tetrahydrocannabinolic acid (Δ8-THCA); delta-8-tetrahydrocannabinol (Δ8-THC)—similar to THC (less potent); 6) Cannabicyclol class: cannabicyclic acid (CBLA); cannabicyclol (CBL); cannabicyclovarin (CBLV); 7) Cannabielsoin class: cannabielsoic acid A (CBEA-A); cannabielsoic acid B (CBEA-B); cannabielsoin (CBE); cannabinol (CBN)—sedative, antibiotic, anticonvulsant, anti-inflammatory; cannabinol methylether (CBNM); cannabinol-C4 (CBN-C4); cannabivarin (CBV); cannabinol-C2 (CBN-C2); cannabiorcol (CBN-C1); cannabinadiol (CBND); cannabinodivarin (CBVD); 8) Cannabitriol class: cannabitriol (CBT); 10-ethoxy-9-hydroxy-delta-6a-tetrahydrocannabinol; 8, 9-dihydroxy-delta-6a-tetrahydrocannabinol; cannabitriolvarin (CBTV); ethoxy-cannabitriolvarin (CBTVE); 8) Miscellaneous cannabinoids class: dehydrocannabifuran (DCBF); cannabifuran (CBF); cannabichromanon (CBCN); cannabicitran (CBT); 10-oxy- delta-6a-tetrahydrocannabinol (OTHC); delta-9-cis-tetrahydrocannabinol (cis-THC); 3,4,5, 6-tetrahydro-7-hydroxy-alpha-alpha-2-trimethyl-9-n-propyl-2, 6-methano-2H-1-benzoxocin-5-methanol (OH-iso-HHCV); cannabiripsol (CBR); trihydroxy-delta-9-tetrahydrocannabinol (triOH-THC).

Another plant proposed in certain embodiments of this invention is Mitragyna speciose. It is an evergreen tree of the coffee (Rubiaceae) family native to Indonesia, Malaysia, Myanmar, Papua. New Guinea, Indonesia, and Thailand. It is best known for generating leaves that contain more than 40 distinct psychoactive compounds. Mitragyna speciosa plant is a 4 to 16-meter-high tropical tree indigenous to South East Asia but now cultivated elsewhere. In Thailand, the tree and leaf preparations are called Kratom. Traditionally, fresh or dried Kratom leaves are chewed or made into a tea; they are seldom smoked. At a low dose, Kratom has stimulating effect and it is used to combat fatigue during long working hours. At high doses it has a sedative-narcotic effect. It is also used in traditional medicine as opium substitute.

Kratom extract contains multiple alkaloids, where some of them have therapeutic potential. The following list shows some alkaloids present in Kratom and their potential therapeutic effect: ajmalicine (raubasine)—cerebrocirculant, antiaggregant, anti-adrenergic (at alpha-1), sedative, anticonvulsant, smooth muscle relaxer (found in Rauwolfia serpentine); ciliaphylline—antitussive, analgesic <1% of total alkaloid content found in Kratom leaf, corynantheidine—μ-opioid antagonist (also found in Yohimbe) <1% of total alkaloid content found in Kratom leaf, corynoxeine—calcium channel blocker <1% of total alkaloid content found in Kratom leaf, corynoxine—dopamine mediating anti-locomotives <1% of total alkaloid content found in Kratom leaf, epicatechin—antioxidant, antiaggregant, antibacterial, antidiabetic, antihepatitic, anti-inflammatory, anti-leukemic, antimutagenic, antiperoxidant, antiviral, potential cancer preventative, alpha-amylase inhibitor (also found in dark chocolate); 9-hydroxycorynantheidine—partial opioid agonist; 7-hydroxymitragynine—analgesic, antitussive, antidiarrheal; primary psychoactive in Kratom, approximately 2% of total alkaloid content found in Kratom leaf, isomitraphylline—immune-stimulant, anti-leukemic <1% of total alkaloid content found in Kratom leaf, isomitrafoline <1% of total alkaloid content found in Kratom leaf, isopteropodine—immuno-stimulant; isorhynchophylline—immuno-stimulant <1% of total alkaloid content found in Kratom leaf, isospeciofoline: <1% of total alkaloid content found in Kratom leaf, mitraciliatine <1% of total alkaloid content found in Kratom leaf, mitragynine—indole alkaloid, analgesic, antitussive, antidiarrheal, adrenergic, antimalarial, possible psychedelic (5-HT2A) antagonist, approximately 66% of total alkaloid content found in Kratom leaf, mitragynine oxindole B <1% of total alkaloid content found in Kratom leaf, mitrafoline <1% of total alkaloid content found in Kratom leaf, mitraphylline—oxindole alkaloid, vasodilator, antihypertensive, muscle relaxer, diuretic, antiamnesic, anti-leukemic, possible immunostimulant <1% of total alkaloid contents in Kratom leaf, paynantheine—indole alkaloid, smooth muscle relaxer, 8.6% to 9% of total alkaloid contents in Kratom leaf, rhynchophylline—vasodilator, antihypertensive, calcium channel blocker, antiaggregant, anti-inflammatory, antipyretic, anti-arrhythmic, antithelmintic <1% of total alkaloid content found in Kratom leaf, speciociliatine—weak opioid agonist, 0.8% to 1% of total alkaloid content of Kratom leaf unique to Kratom; speciogynine—smooth muscle relaxer, 6.6% to 7% of total alkaloid contents of Kratom leaf, speciophylline—indole alkaloid, anti-leukemic <1% of total alkaloid contents of Kratom leaf; tetrahydroalstonine—hypoglycemic, anti-adrenergic (at alpha-2).

Another plant type proposed in some of the embodiments presented here is the genus Psychotria. It is one of the largest genera of flowering plants and the largest within Rubiaceae—estimated 1000 to 1650 species distributed worldwide (Porto, Henriques, & Fett-Neto, 2009). Psychotria species contains indole alkaloids and may yield certain bioactive extracts. The examples include antibiotic activity in extracts from Psychotria microlabastra and Psychotria capensis (Africa), antiviral activity of Psychotria serpens (China), and antiviral/antifungal and antiinflammatory activities found in Psychotria hawaiiensis and Psychotria insularum (Central America) (Porto, Henriques, & Fett-Neto, 2009).

Several South American Psychotria species are used as medicinal plants by Amazon native populations. Active molecules produced by Psychotria species include naphtoquinones, peptides, benzoquinones, pigments and alkaloids (Porto, Henriques, & Fett-Neto, 2009). The extracts of Psychotria colorata show analgesic activity, and preliminary tests point to alkaloids being responsible for the effect (Elisabetsky, Amador, Albuquerque, Nunes, & Carvalho, 1995). Interesting enough, in the genus Psychotria, analgesic activity was identified in alkaloids with diverse molecular structures, such as the indole monoterpene-type alkaloid umbellatine, and the pyrrolidinoindoline-type alkaloids hodgkinsine, psychotridine, isopsychotridine A, isopsychotridine B, quadrigemine C, calycanthine, isocalycanthine, among others (Both, Kerber, Henriques, & Elisabetsky, 2002) (Amador, Elisabetsky, & Onofre de Souza, 1996) (Amador T. A., Verotta, Nunes, & Elisabetsky, 2001). The most significant to date are hodgkinsine, which acts as both a mu-opioid agonist and NMDA antagonist, (Amador T. A., Verotta, Nunes, & Elisabetsky, 2001) and psychotridine which is an NMDA antagonist with little or no mu-opioid affinity (Amador T. A., Verotta, Nunes, & Elisabetsky, 2001).

This invention generally relates to pharmaceutical and nutraceutical compounds and methods for reducing inflammation, blood sugar, gastric acid secretion, symptoms of allergy, common cold and flu, as well as treating infections and pain associated with trauma, medical procedure, and diseases and disorders in subjects in need thereof, as well as a method of administering therapeutically-effective amount of said compounds containing certain natural and/or synthetic Picralima nitida and/or Vinca major and/or Cannabis sativa and/or Mitragyna speciose, and/or Psychotria genus alkaloids and/or their derivatives/analogs and other substances.

Other medical conditions are also contemplated by this invention that include, but are not limited to: upper respiratory tract infection (common cold) or an acute respiratory illness (flu) of viral or bacterial origin, acid refluxing, phytosis and mycosis, gastric and duodenal ulcers, Huntington' s Disease; Wilson's Disease; Parkinson's Disease; chronic cough; cough associated with Asthma, allergic reaction, respiratory disease, gastro-oesophageal reflux disease (GORD) and post nasal drip syndrome (PNDS); obesity and body weight reduction; diabetes; metabolic and endocrine diseases and disorders; autoimmune system responses (allergic reactions), athetosis-related to damage or degeneration of basal ganglia; gastritis; acid overproduction; acid reflux; peptic ulcers; minor tranquilizers, alcohol, cocaine, (meta)amphetamine, and opioid withdrawal syndromes; symptoms or side effects associated with anti-retroviral therapy, chemotherapy and radiation therapy; AIDS; rheumatoid arthritis; osteoarthritis; fibromyalgia; pain and spasticity symptoms associated with MS, Neuromuscular Junction Disorder, autoimmune diseases and disorders, motor neuron diseases and disorders, neurodegenerative diseases and disorders; pain associated with cancer; trauma; athletic performance; migraine; surgical intervention or medical treatment; stroke; heart attack; dental and gum pain; abdominal pain; bone pain, muscle pain; neurological pain; stomach ulcers-related pain; gallbladder disease-related pain; Central Pain Syndrome (CPS); sports trauma; chronic pain disorder (nociceptive pain, neuropathic pain, chronic back or leg pain, painful neuropathies, Complex Regional Pain Syndrome), and acute pain.

Extracts of the seeds of Picralima nitida and Vinca major have been reported to have opioid analgesic activity. An isolated tissue bioassay and radioligand binding assays have been performed by Menzies et al., (1998) to determine the opioid activity of five alkaloids that may be found Picralima nitida: akuammidine, akuammine, akuammicine, akuammigine and pseudoakuammigine. The data show that some of these alkaloids extracted from the plant possess varying degrees of agonist and antagonist activity at opioid receptors but have neither high affinity nor selectivity for mu, delta or kappa-opioid receptors or the ORL1-receptor (Menzies, Paterson, Duwiejua, & Corbett, 1998). Likewise, several alkaloids were isolated from Vinca major plant: reserpinine, majoridine, vincamajoridine, vincamine, vincamajine, perivincine, pubescine, vinine, vincawajine, majorinine, 10-Methoxyvinorine (2016) and some of them also have opioid analgesic activity.

Besides the pain relief, both Picralima nitida and Vinca major reportedly can treat chest and stomach problems, as well as pneumonia and intestinal worms. In the latter case, the seeds or bark of Picralima nitida is crushed or chewed and eaten or a decoction from the roots, seeds or bark is consumed. In Nigeria, a decoction of the leaves is taken by mouth or used as a lotion against measles (Igwe & Mgbemena, 2014). In Ghana, dry Picralima nitida leaves are boiled in water and taken to treat guinea worms; while in Cameroon a fruit decoction is taken to cure cough or typhoid fever (Igwe & Mgbemena, 2014). Picralima nitida leaves have been reported to possess both antidiabetic and antioxidant properties (Teugwa, Mejiato, Zofou, Tchinda, & Boyom, 2013), while the seeds contain a mixture of alkaloids producing antipyretic and anti-inflammatory effects along with analgesia (Duwiejua, Woode, & Obiri, 2002) (Lewin, Le Ménez, Rolland, Renouard, & Giesen-Crouse, 1992). Antidiarrhoeal activity of the fruit-rind of Picralima nitida has also been reported and shown to have the activity due to its inhibitory effect on gastrointestinal propulsion (Mabeku, et al., 2006). Also, the antiplasmodial activity of ethanolic seed extract of Picralima nitida has been reported (Okokon, Antia, Igboasoiyi, Essien, & Mbagwu, 2007). The plant was also investigated experimentally to exhibit antimicrobial, antipyretic and anti-inflammatory activities (Fakeye, Itiola, & Odelola, 2000). It has been demonstrated that Picralima nitida has a broad activity for treating parasitic diseases, which lends credibility for its use against diarrhoea, gonorrhoea and intestinal worms (Fakeye, Itiola, & Odelola, 2000) (Mabeku, et al., 2006).

In some embodiments of the proposed invention, CBs can be utilized to increase medicinal efficacy in certain clinical applications. CBs are a group of chemicals known to activate CB receptors in cells. These chemicals, which are found in cannabis plants, are also produced endogenously in humans and animals, these are termed endocannabinoids. There are also synthetic CBs that are chemicals with similar structures to plant CBs or endocannabinoids. Plant cannabinoids can also be isolated such that they are “essentially pure” compounds. These isolated CBs are largely free of other naturally occurring compounds, such as other minor CBs and molecules.

The primary CB receptor subtypes are CB receptors type 1 (CB1) and type 2 (CB2). CB1 receptors are highly expressed in the Central Nervous System (CNS), especially the basal ganglia, and also identified in almost all peripheral tissues and cell types. CB2 receptors are expressed primarily in the immune system, where they modulate inflammation, but are also expressed in the CNS, particularly in neurons within the dorsal vagal motor nucleus, the nucleus ambiguous, the spinal trigeminal nucleus, and microglia. CB2 receptors were also found in the basal ganglia and studies suggest that impairment of these receptors may be associated with dyskinesia. While most actions of CBs are related to CB1 and CB2 receptors, other receptor types have been described, including the Transient Receptor Potential Vanilloid type 1 (TRPV1) cation channel, the GTP-binding Protein-coupled Receptor GPR55, the abnormal-CBD receptor, and the Peroxisome-Proliferator-Activated Receptor (PPAR) (Kluger, Triolo, Jones, & Jankovic, 2015).

Endogenously produced CBs (eCBs) are lipophilic compounds that demonstrate varying degrees of affinity for G-protein coupled CB receptors and include anandamide and 2-arachidonoglycerol. eCBs primarily function through retrograde signaling, wherein post-synaptic activity leads to eCB production and release with backward transmission across the synapse to depress presynaptic neurotransmitter release. The Endo-Cannabinoid System (ECS) may also support synapse formation and neurogenesis. Within the basal ganglia, eCBs and CB1 receptors tend to increase GABAergic and inhibit glutamatergic transmission eCBs also tend to inhibit dopamine release through GABAergic mechanisms. eCBs are not stored and are quickly degraded after exerting a transient and localized effect. Removal of eCBs from the extracellular space occurs through cellular uptake and metabolism with anandamide degraded primarily by Fatty Acid Amide Hydrolysis (FAAH) and 2-AG degraded by monoacylglycerol lipase. (Kluger, Triolo, Jones, & Jankovic, 2015)

The disclosed invention finds that, in one embodiment, a number of alkaloids contained in Picralima nitida or Vinca major and Cannabis sativa plants, when combined, could be used as a substitute for diclofenac or indomethacin, or even tramadol or morphine, having a substantial analgesic action, and can provide together with cannabinoids a sedative and muscle relaxant effects akin to the effects of benzodiazepines. In another embodiment, Picralima nitida or Vinca major can be used without the cannabinoids to relieve pain, inflammation, joint stiffness, such as when caused by arthritis, and other symptoms of diseases and disorders. Akuammine, vincamajoridine, dihydroakuammine, akuammidine and pseudoakuammigine, found in Picralima nitida or Vinca major, as well as tetrahydrocannabinol (THC) and cannabidiol (CBD), found in Cannabis sativa, are the two groups of alkaloids mainly responsible for the analgesic, antitussive and anti-inflammatory effects. Further analgesic and antitussive effects can be achieved with addition of umbellatine or hodgkinsine alkaloids derived from the genus Psychotria, such as Psychotria oleoides, Psychotria beccaroides, Psychotria umbellate, Psychotria forsteriana, or Psychotria colorata plants. Even greater analgesic action can be attained with mitragynine, pseudoindoxyl, or 7-hydroxymitragynine derived from Mitragyna speciose.

The results of investigation conducted by Menzies et al., (1998) show that alkaloids from Picralima nitida possess varying degrees of affinity, preference and efficacy for opioid receptors. Akuammidine, akuammine, akuammicine and pseudoakuammigine bind with low affinity to mu-, delta- and kappa-opioid binding sites, but akuammigine showed no opioid binding (Menzies, Paterson, Duwiejua, & Corbett, 1998). The opioid affinities of these alkaloids are at least two orders of magnitude less than those of the selective mu-opioid receptor ligand DAMGO, the selective delta-opioid receptor ligand DPDPE, and the selective kappa-opioid receptor ligand CI-977 (Menzies, Paterson, Duwiejua, & Corbett, 1998). For practical purposes, a selective opioid compound is at least 100 times more active at its preferred site than at other opioid binding sites, i.e., it has a relative affinity of >0.98. Menzies et al., (1998) concluded that none of the alkaloids from Picralima nitida are selective. Although akuammidine and akuammine show a preference for mu-opioid binding sites, and akuammicine for kappa-opioid binding sites, the affinity for their preferred site, though, is less than 10-fold greater than that for another opioid site (Menzies, Paterson, Duwiejua, & Corbett, 1998).

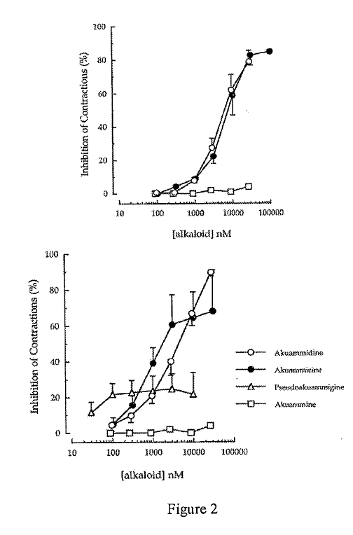

The effects of alkaloids extracted from Picralima nitida on the electrically-evoked contractions of the guinea pig myenteric plexus—longitudinal muscle preparation and of the mouse isolated vas deferens are shown in FIG. 2 (Menzies, Paterson, Duwiejua, & Corbett, 1998). It is, however, well-recognized that neurotransmission in some bioassay preparations is sensitive to inhibition by opioids. In the research conducted by Menzies et al., (1998) akuammicine, akuammidine, and pseudoakuammigine, each inhibited the electrically-induced contractions of guinea pig myenteric plexus—longitudinal muscle preparations. These inhibitions were antagonised by naloxone confirming an action at opioid receptors. According to Menzies et al., (1998), it seems likely that the alkaloids are acting pre-junctionally on opioid receptors to inhibit neurotransmitter release as they did not affect contractions caused by carbachol stimulating post-junctional muscarinic cholinoceptors.

It is peculiar, however, that one of the main and most abundant alkaloids that is believed to be greatly attributing to the analgesic actions of the Picralima nitida extract, akuammine, causing pain relief by an agonist action, most likely at mu-opioid receptors, was found by Menzies et al., (1998) to be an opioid antagonist. Although akuammine is not an agonist at opioid receptors, a metabolite may well be according to Menzies et al., (1998), and it is the metabolite which exerts the analgesic action. Another explanation for the analgesic activity of akuammine is that it blocks the action of a pronociceptiverhyperalgesic endogenous substance, i.e., which produces hypersensitivity to pain (Menzies, Paterson, Duwiejua, & Corbett, 1998). Nociceptinrorphanin FQ acting at the ORL receptor has been proposed to be a such substance (Menzies, Paterson, Duwiejua, & Corbett, 1998). The analgesic actions of akuammine, therefore, could result from antagonism of the hyperalgesic activity of nociception/orphanin FQ. In binding assays, however, according to Menzies (1998) neither akuammine nor any of the other alkaloids showed appreciable affinity for ORL1-binding sites. In addition, the bioassay data, according to Menzies (1998), indicates that akuammine does not antagonize nociceptin at functional ORL1 receptors in the guinea pig small intestine. Thus, Menzies et al., (1998) conclude that akuammine does not produce analgesia by antagonising the actions of nociception/orphanin FQ at ORL1 receptors.

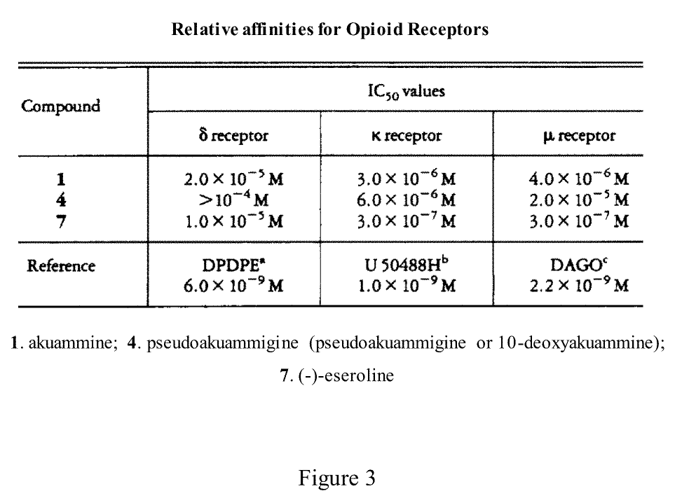

In another research, conducted by Guy Lewin et al., (1992) the binding assays were performed using membranes prepared from rat central nervous system, and after the incubation period, bound and unbound radioligands were separated by rapid filtration, and radioactivity bound to membranes in the absence and presence of unlabeled compounds was counted in a beta-scintillation counter. Specific binding and displacement were then established. This research, however, demonstrated that akuammine had micromolecular affinities for kappa and mu receptors, but its affinity for the delta opioid receptor was ten times lower (Lewin, Le Ménez, Rolland, Renouard, & Giesen-Crouse, 1992). The affinities of three opioid receptor subtypes of the following compounds were investigated as further shown in the FIG. 3: akuammine, pseudoakuammigine (pseudoakuammigine or 10-deoxyakuammine) and (−)-eseroline (Lewin, Le Ménez, Rolland, Renouard, & Giesen-Crouse, 1992).

In common preclinical practice, however, it is often not akuammine which is taken for pain relief but rather extracts of Picralima nitida seeds. The results obtained by Dapaah et. al., (2016) from the BALB/c mice tail flick test, showed significant antinociceptive effect at the doses of extract 100-500 mg/kg. This was also observed for both diclofenac and morphine (Dapaah, Koffuor, Mante, & Ben, 2016). The tail flick test is considered to induce a spinal reflex, but it could also involve higher neural structures and so the method essentially identifies the centrally-acting analgesics. Thus, the extract can be said to be acting through a centrally mediated pathway by elevating pain threshold of animals towards heat. The writhing test, however, helps identifying peripheral analgesic compounds as well as the central.

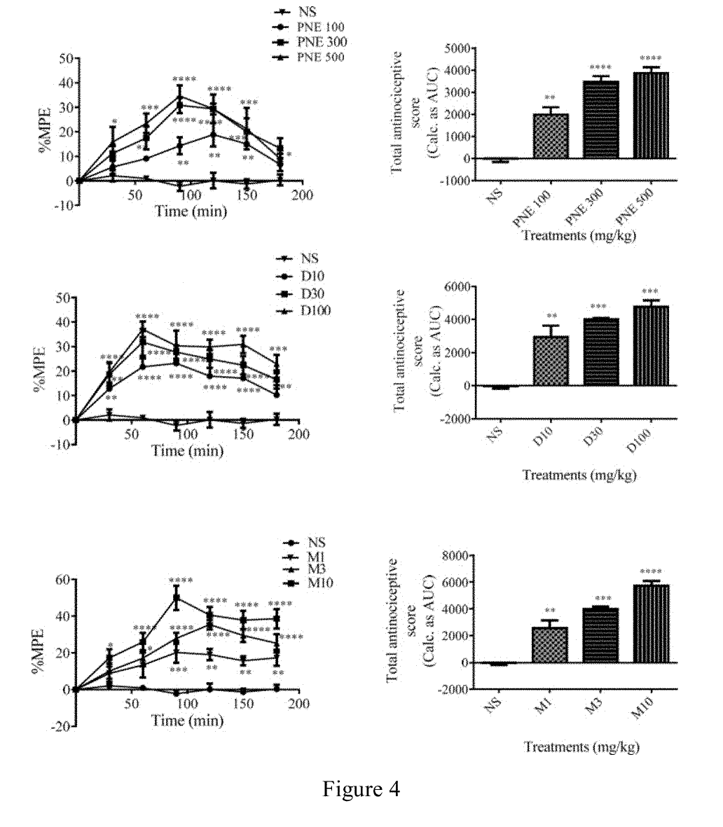

According to Dapaah et. al., (2016) both the extract and the reference drugs caused increase in the tail withdrawal latency, compared to the control (see FIG. 4) (Dapaah, Koffuor, Mante, & Ben, 2016). According to Dapaah et. al., (2016), the extract (100-500 mg/kg) dose dependently increased (P<0.0001) tail withdrawal latencies; diclofenac (10-100 mg/kg) likewise exhibited increased tail withdrawal latencies (P=0.0002); and morphine (1-10 mg/kg) also showed a significant (P<0.0001) dose dependent increase in tail withdrawal latencies.

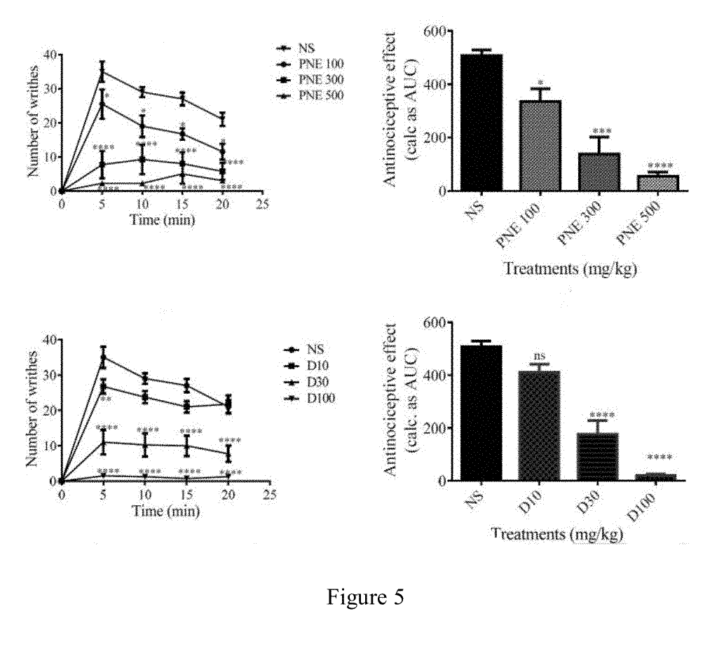

According to Dapaah et. al., (2016) the acetic acid-induced writhing assay, the extract (100, 300, 500 mg/kg) and diclofenac (10, 30, 100 mg/kg) suppressed the writhing (FIG. 5). The extract has significantly dose-dependently reduced abdominal writhes over the 20 minutes observation (P<0.0001), as well as diclofenac (P<0.0001) (Dapaah, Koffuor, Mante, & Ben, 2016).

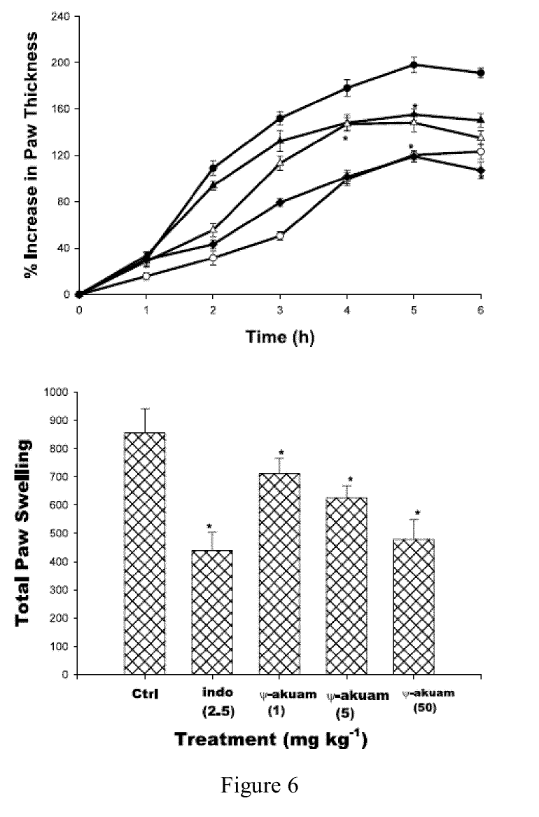

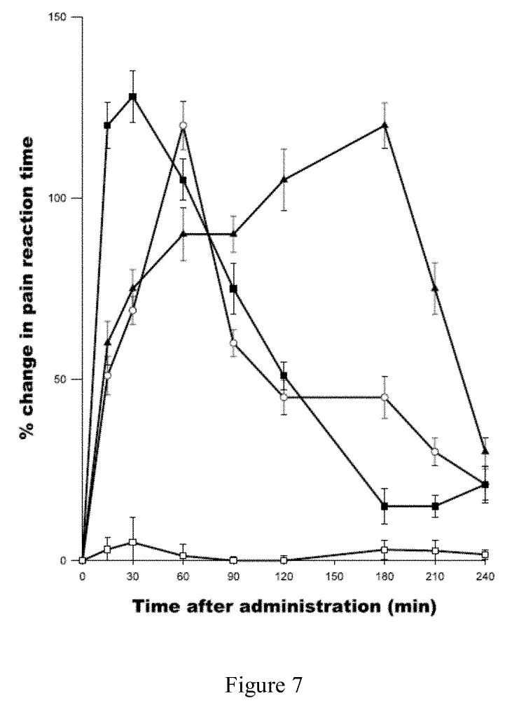

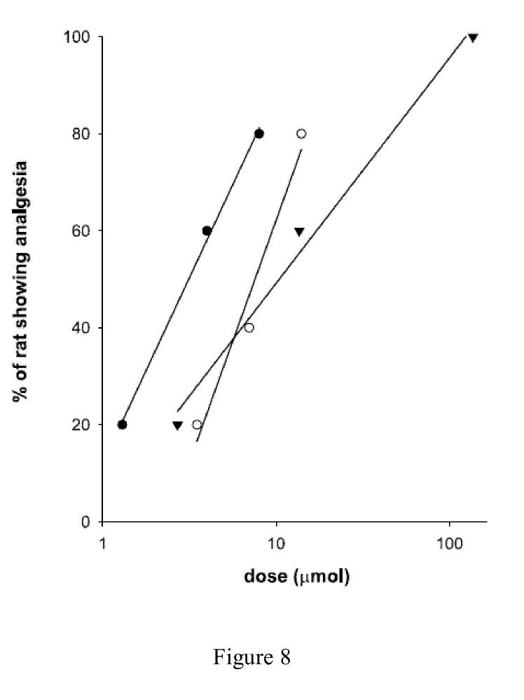

Duwiejua et al., (2002) have investigated one of the Picralima nitida alkaloids, pseudoakuammigine. The alkaloid was tested for anti-inflammatory and analgesic actions in rats. The test had revealed a dose-dependent inhibitory activity on carrageegeenan-induced rat paw oedema (see FIG. 6) (Duwiejua, Woode, & Obiri, 2002). The analgesic effect was further demonstrated in the aforesaid study, where the morphine peaked after 30 min, whilst pseudoakuammigine and indomethacin (another anti-inflammatory drug) peaked at 180 and 60 minutes respectively (see FIG. 7) (Duwiejua, Woode, & Obiri, 2002). The research has shown that on molar basis, pseudoakaummigine was 3.5 times and 1.6 times less potent than morphine and indomethacin respectively as an analgesic, where the ED50 values were 2.9, 10 and 6.3 muM for morphine, pseudoakaummigine and indomethacin respectively (see FIG. 8); naloxone had significantly (P<0.05) antagonized the analgesic actions of both morphine and pseudoakaummigine (Duwiejua, Woode, & Obiri, 2002).

Duwiejua et al., (2002) concluded that the mechanism of pseudoakuammigine's action is not known but the results indicate that pseudoakuammigine is a potentially effective anti-inflammatory agent, at least in the early exudative phase. Duwiejua et al., (2002) had established a direct correlation between the dose and anti-inflammatory effect of pseudoakaummigine in Wistar rats. The maximal inhibitory effect was attained at 50 mg kg, p.o. was 44%. Attenuation of the pseudoakuammigine analgesic action by naloxone indicated an interaction with opioid receptors in vivo, also showing the lack of specificity for opioid receptor subtypes which is a property shared with other Picralima nitida alkaloids (Duwiejua, Woode, & Obiri, 2002). Duwiejua et al., (2002) further concluded that in spite of the evidence that pseudoakuammigine has marked analgesic actions mediated via interaction with opioid receptors, the evidence is insufficient to exclude the involvement of analgesic effect mediated via interactions with peripheral non-opioid receptors. Also, the greater potency of morphine in comparison with pseudoakuammigine can be attributed to the difference in routes of administration (Duwiejua, Woode, & Obiri, 2002).

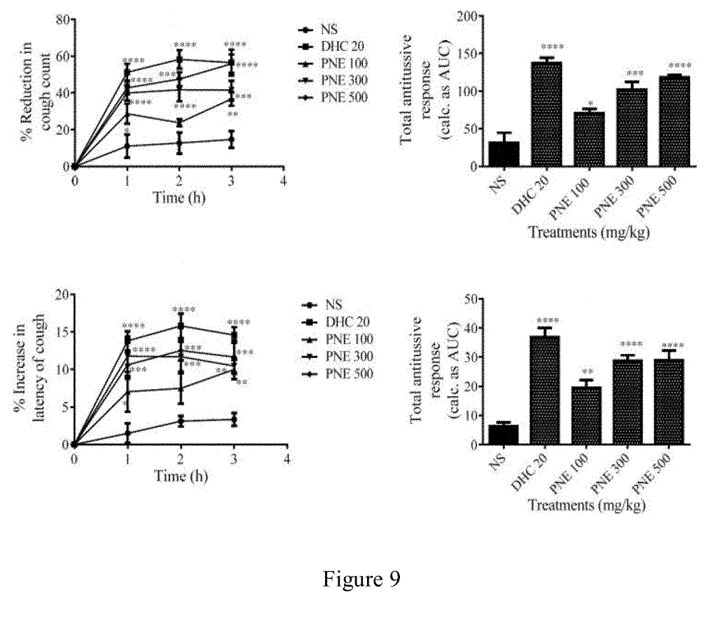

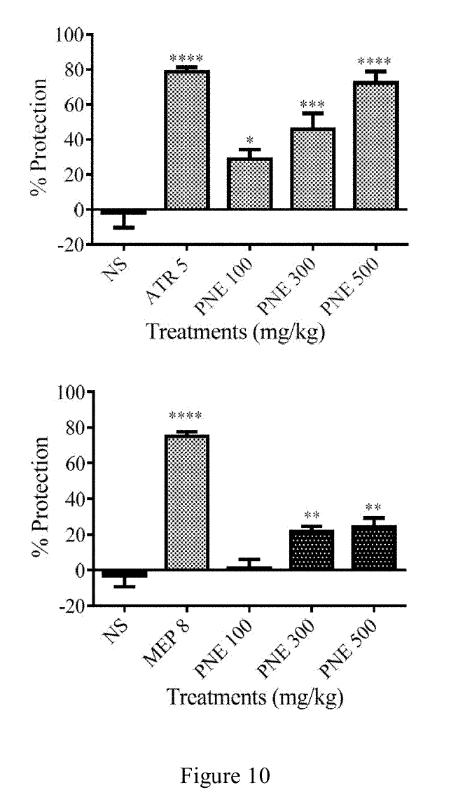

In addition, Picralima nitida and Vinca major extracts exhibit antitussive and expectorant effects that are comparable to those of codeine in animal studies, as well as antibacterial and anti-allergy effects such as mast cells stabilization, mucus suppression, antipyretic, bronchodilator effects, and somewhat anxiolytic action (Dapaah, Koffuor, Mante, & Ben, 2016) (Erharuyi, Falodun, & Langer, 2014). Dapaah et. al., (2016) have demonstrated significant antitussive effects where both dihydrocodeine and Picralima nitida extract (100, 300, and 500 mg/kg) dose-dependently reduced (P<0.05) cough count, and increased significantly (P<0.01) the latency of cough (see FIG. 9). Atropine and Picralima nitida extract (100, 300, 500 mg/kg) significantly (P<0.05-0.0001) protected in the animal studies against bronchoconstriction and cough induced by acetylcholine (see FIG. 10) (Dapaah, Koffuor, Mante, & Ben, 2016). The percentage protection produced by Picralima nitida extract was dose-dependent. Similarly, mepyramine and Picralima nitida extract (300, 500 mg/kg) reduced significantly (P<0.01-0.0001) bronchoconstriction and cough induced by histamine (see FIG. 10) (Dapaah, Koffuor, Mante, & Ben, 2016).

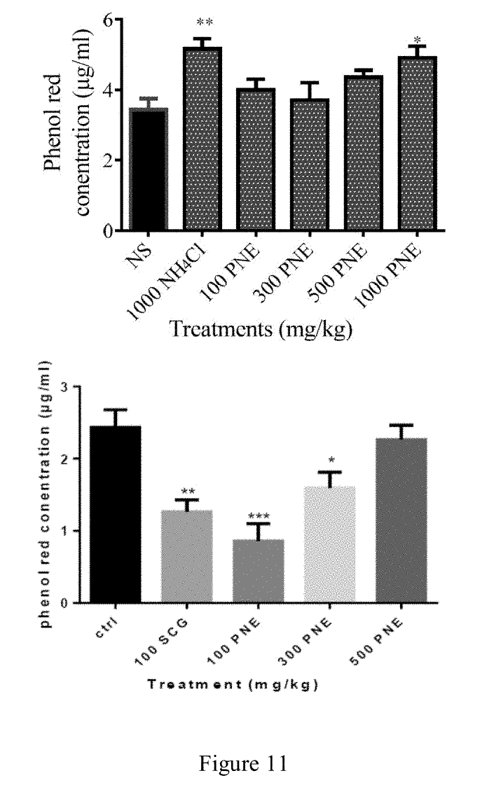

Picralima nitida plant extract, according to Dapaah et. al., (2016) have demonstrated a mild expectorant property at doses of 100, 300, 500 mg/kg (P>0.05) in tracheal phenol red secretion, compared to the control. However, the 1000 mg/kg dose exhibited a significant effect (P<0.05), although lesser in magnitude than that for ammonium chloride (P<0.01), the reference expectorant drug (see FIG. 11) (Dapaah, Koffuor, Mante, & Ben, 2016). Another comparison was made with sodium cromoglicate that also caused a reduction (P<0.01) in tracheal phenol red secretion.

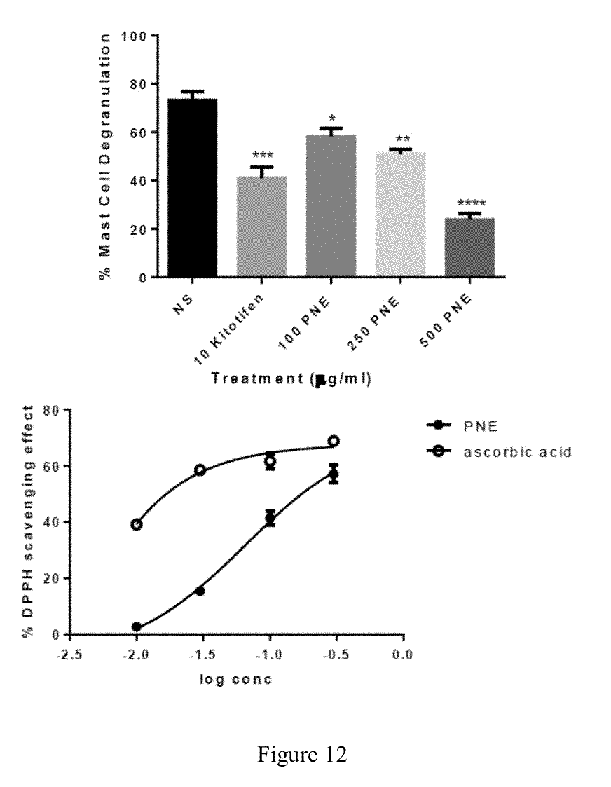

In another experiment Picralima nitida extract has demonstrated mast cell stabilizing effect. Treatments with the extract and sodium cromoglicate were able to reduce significantly (P<0.01) mast cell degranulation induced by the compound 48/80 relative to the control (see FIG. 12) (Dapaah, Koffuor, Mante, & Ben, 2016). Picralima nitida plant extract also demonstrates free radical scavenging ability—marked antioxidant properties. The EC50 obtained for Picralima nitida seed extract was 0.06530 mg/ml and that for the reference antioxidant, ascorbic acid was 0.001070 mg/ml (see FIG. 12) (Dapaah, Koffuor, Mante, & Ben, 2016). The result of the 1, 1-Diphenyl-2-picrylhydrazyl (DPPH) radical scavenging assay in another study conducted by Erharuyi et. al, (2012) showed that Picralima nitida root bark extracts have appreciable DPPH scavenging effect with the crude extract being the most active. The extracts, especially the crude and the ethyl acetate fraction, have the scavenging effects comparable to that of ascorbic acid (the standard antioxidant) but these, however, were not statistically significant (Erharuyi & Falodun, 2012). Erharuyi et. al, (2012) had also found a dose dependent radical scavenging activity with the Picralima nitida root bark extract.

In another study conducted by Fakeye et al., (2000) the in vitro antioxidant activities of methanol and hydroethanol extracts of the stem bark and leaves of Picralima nitida were evaluated by the DPPH free radical-scavenging method and the effect of extracts treatment on selected oxidative stress markers: malondialdehyde, hydrogen peroxides and catalase were also evaluated in mice. The extracts exhibited good free radical scavenging activity and extracts treatment resulted in a significant reduction in malondialdehyde and hydrogen peroxide levels as well as a marked increase in catalase activity (Fakeye, Itiola, & Odelola, 2000). The antioxidant capacity of ethanol, ether, ethyl acetate, butanol and aqueous extracts of Picralima nitida seeds were determined using free radical induced hemolysis in red blood cells. In the study, it was noted that Picralima nitida seed extract have good antioxidant capacity with the butanol extract exhibiting the highest activity (Shittu, Gray, Furman, & Young, 2010). Similarly, the antioxidant activity of methanol root bark extract of Picralima nitida has been investigated in vitro using the DPPH free radical scavenging method. The study revealed that the extract exhibited appreciable percentage of radical scavenging activities with IC50 value of 5 mu g/mL (Erharuyi & Falodun, 2012).

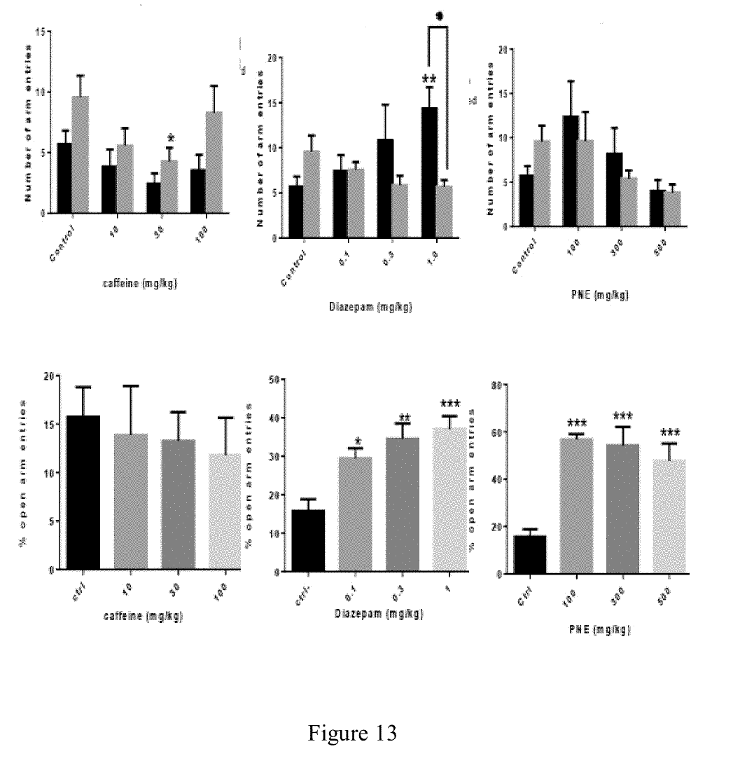

Picralima nitida seed extract, according to Dapaah et. al., (2016) also demonstrates a mild anxiolytic effect. The extract (100, 300, 500 mg/kg) enhanced, dose-independently, activities of mice in the open arm test by increasing percentage of entry into open arms (F3, 19=12.97, P<0.0001) and percentage of time spent in open arms (F3, 19=2.148, P=0.1278) (see FIG. 13) (Dapaah, Koffuor, Mante, & Ben, 2016). There was also a reduction in risk assessment by decreasing both the percentage of protective head dips (F3, 19=6.635, P=0.0030) and percentage of protective stretch attend postures (F3, 19=2.552, P=0.0860) (Dapaah, Koffuor, Mante, & Ben, 2016). Nevertheless, diazepam (0.1-1 mg/kg) dose dependently and significantly increased the percentage of open arm entries (F3, 22=8.677, P=0.0005) and percentage of time spent in open arm (F3, 22=9.085, P=0.0004). Diazepam also reduced risk assessment by decreasing both the percentage of protective head dips (F 3, 22=10.43, P=0.0002) and the percentage of protective stretch attend postures (F3, 22=4.071, P=0.0192). These effects confirmed the anxiolytic effects of both Picralima nitida seed extract and diazepam (Dapaah, Koffuor, Mante, & Ben, 2016).

With regard to caffeine, according to Dapaah et. al., (2016) it increased open arm avoidance by reducing the percentage of open arm entries (F3, 21=0.1934, P=0.8997) and percentage of time spent in open arms (F3, 21=3.673, P=0.0285). Caffeine also increased both the percentage of protective head dips (F 3, 21=0.4492, P=0.7205) and percentage of protective stretch attend postures (F3, 23=3.764, P=0.0247)—all indicative of anxiogenic effect of caffeine (Dapaah, Koffuor, Mante, & Ben, 2016). Further, Picralima nitida seed extract (100, 300, 500 mg/kg) dose-independently increased the frequency of central zone entries, time spent in a central zone, percentage entry into a central zone (F 3, 18=4.216, P=0.0201), and percentage time spent in a central zones (F3, 18=3.337, P=0.0427) (Dapaah, Koffuor, Mante, & Ben, 2016). These observations support the claim that Picralima nitida seed extract acts as an anxiolytic. The reference anxiolytic drug, diazepam (0.1-1 mg/kg), according to Dapaah et. al., (2016) also dose dependently increased the frequency of central zone entries, time spent in a central zone, percentage of entries into a central zone (F 3, 26=6.318, P=0.0023) and the percentage time spent in a central zone (F3, 26=2.793, P=0.0603).

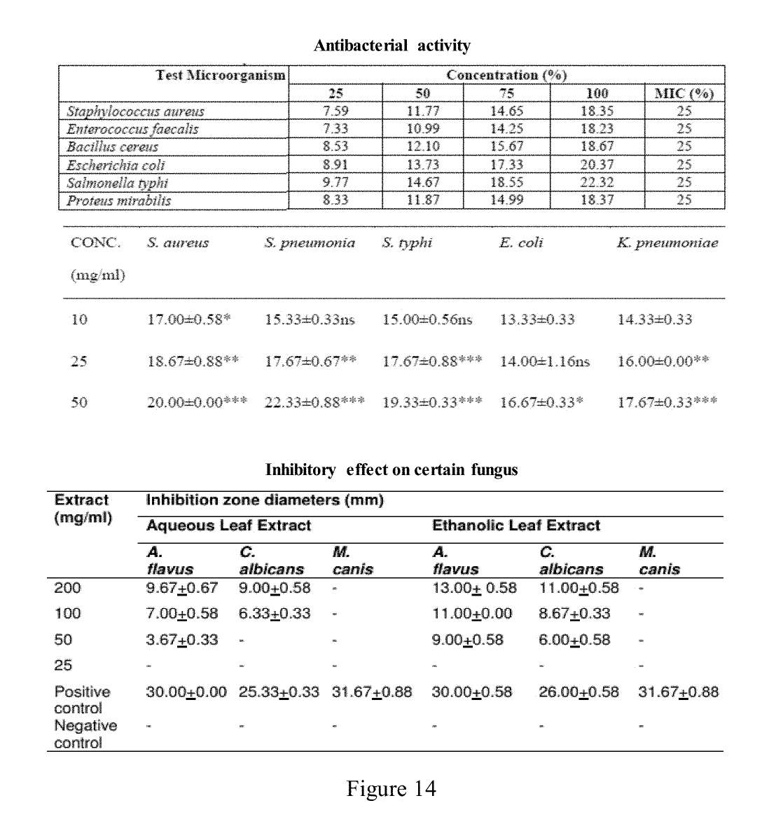

Picralima nitida plant extract, according to Erharuyi et al., (2014) also demonstrates strong antibacterial and antifungal properties. According to Igwe et al., (2014) the Picralima nitida leaves extract exhibited marked antibacterial activity against the six pathogens tested (in the order of activity): Salmonella typhi, Escherichia coli, Bacillus cereus, Proteus mirabilis, Staphylococcus aureus, Enterococcus faecalis (see FIG. 14). The minimum inhibitory concentration (MIC) of the leaf extract was 25-100%. From the results, greater antibacterial activity was shown against Salmonella typhi and Escherichia coli, suggesting that the extract could be used in the treatment of typhoid fever, bloody diarrhea, urinary tract infections, severe anemia and kidney failure (Igwe & Mgbemena, 2014).

In another study, according to Dapaah et. al., (2016) Picralima nitida seed extract showed antibacterial activity at concentrations 5-50 mg/ml on Salmonella typhi, Streptococcus pneumonia, and Staphylococcus aureus. The lowest effect was against Salmonella typhi at 5 mg/ml with a zone of 13.0±0.00 mm whiles the highest response was observed against Streptococcus pneumonia at 50 mg/ml with a zone of 22.3±0.88 mm (see FIG. 14). Further, fungal species used in the study conducted by Ubulom et al., (2012) exhibited varying degrees of susceptibility to the leaf extracts of Picralima nitida (see FIG. 14). Results obtained revealed that both the aqueous and ethanolic leaf extracts exerted antifungal effect on Aspergillus flavus and Candida albicans, but no antifungal effect was exhibited against Microsporum canis at the extract concentrations used (Ubulom, Imandeh, Udobi, & IIya, 2012). However, it was reported that flavonoids, which possess antifungal property, were not present in the studied extract. Thus, the absence of flavonoids and tannins in the leaves may have been the reason for the absence of inhibitory effect of the leaf extracts on Microsporum canis (Ubulom, Imandeh, Udobi, & 2012).

The antipyretic activity of Picralima nitida fruit has also been established. The result of the study showed that the methanol fruit extract at a dose of 50 mg/kg produced a mean percentage antipyrexia of 38.7% on lipopolysaccharide-induced pyrexia in rabbits, which was comparable to aspirin (29.0% at 200 mg/kg) (Ezeamuzie, Ojinnaka, Uzogara, & Oji, 1994).

In another study, an antiulcer activity was evaluated. The antiulcer activity of the methanol extract and chloroform/methanol fractions of Picralima nitida seeds were evaluated using the aspirin-pylorus-ligation method in rats. The study revealed that the extract and fractions of Picralima nitida seeds produced significant (P<0.05) reduction of ulcer index, total acidity, pepsin activity and increase in mucoprotective parameters such as phenol red content. The study reports a potent antiulcer activity with the ulcer inhibition percentage of 56.36%, 40.00% and 56.36% for the methanol extract and chloroform/methanol fraction respectively, compared to the control (normal saline) (Mabeku, Kouam, Paul, & Etoa, 2008).

In another study, performed by Okonta et. al., (2011) oral administration of the methanol extract, chloroform fraction, and methanol fraction at 1,000 mg/kg reduced gastric ulcer in aspirin ligation animal model by 56.4%, 40.0% and 56.3%, respectively; and the fractions of the extract significantly (P<0.05) reduced gastric emptying time when compared to the control. Gastric acidity was significantly decreased when compared with the saline group, 40.25 mEq/L in methanol extract, 50.0 mEq/L in chloroform fraction, 51.25 mEq/L in methanol fraction, but had no significant effect on the gastric secretion volume (Okonta, Adibe, & Ubaka, 2011). The fractions and extract exhibited potent antiulcer activity by significantly decreasing ulcer indices. Reduction of ulcers in aspirin ligation model usually signifies a protective action and possibly an anti-secretory effect (Okonta, Adibe, & Ubaka, 2011).

It was further discovered that the extracts of Vinca major and Picralima nitida have several biological activities, which can be beneficial for skin. Non-limiting examples of these biological activities include antioxidant properties and the ability to inhibit melanin production. For instance, Picralima nitida extract may provide: B16 inhibition −23.84, TNF-α inhibition−29.153, and antioxidant activity −42.3.

Overall, Picralima nitida or Vinca major extracts have a variety of physiological effects. Present evidence strongly supports analgesic, anti-inflammatory, as well as antitussive activity. Competition binding assays revealed that four of the five alkaloids extracted from Picralima nitida bound to opioid sites in homogenates of guinea pig brain. It is suggested that both akuammidine and akuammine interact with all three opioid sites but bound preferentially to mu-opioid binding sites with K values of 0.60 and 0.48 mM, respectively (Menzies, Paterson, Duwiejua, & Corbett, 1998). These values correspond to affinities of 1.7 mM⁻¹ for akuammidine and 2.1 mM⁻¹ for akuammine (Menzies, Paterson, Duwiejua, & Corbett, 1998). Other alkaloids, such as akuammicine bounds preferentially to kappa-opioid binding sites with a K value of 0.19 mM. The affinity of akuammicine for kappa-opioid binding sites 5.3 mM⁻¹ was 10-fold greater than for either mu or delta sites reflected in a relative kappa-affinity of 0.86 (Menzies, Paterson, Duwiejua, & Corbett, 1998). Pseudoakuammigine bound equally to both mu and delta-opioid binding sites. Akuammigine showed no affinity for any of the opioid binding sites (Menzies, Paterson, Duwiejua, & Corbett, 1998). Akuammine, akuammidine and pseudoakuammigine displayed little affinity for ORL-binding sites (Menzies, Paterson, Duwiejua, & Corbett, 1998).

Evidence exists that increase in safety and medicinal efficacy may be achieved by combining, for example, the extracts or specific alkaloids of Vinca major, Mitragyna speciosa and Cannabis sativa. In one embodiment, a mixture of a cannabinoid THC and an opiate, such as mitragynine, 7-hydroxymitragynine or akuammine, was administered to a subject in need of pain relief. It is conceivable that when an opiate is administered with a cannabinoid, in one embodiment, THC, the amount of opiate administered could be reduced without diminishing the pain relief. It is also conceivable that addition of CBD in proportions of 1:2 (THC:CBD) may reduce psychotropic effects of THC and prolong its therapeutic activity. CBD—an antagonist of CB1 and CB2 receptors (Pertwee, 2008) is believed to be modulating the THC psychotropic effect (Russo & Guy, 2006) (Karniol, Shirakawa, Kasinski, Pfeferman, & Carlini, 1974).

The therapeutic potential of CBs is well-described in the literature. However, it is worth mentioning here several studies related to analgesia. A study by Formukong et al., (1988) was undertaken to determine the analgesic and anti-inflammatory activity of various CBs and CB pre-cursors. Oral administration of CBD was found to be the most effective at inhibition of phenyl-p-benzoquinone-induced writhing in mice. Formukong et al. (1988) emphasize that with the exception of CBN and delta 1-THC, the cannabinoids and olivetol (their biosynthetic precursor) demonstrated activity in the PBQ test exhibiting their maximal effect at doses of about 100 mg/kg. Delta 1-THC only became maximally effective in doses of 10 mg/kg. This higher dose corresponded to that which induced catalepsy and is indicative of a central action (Formukong, Evans, & Evans, 1988). CNB demonstrated little activity and even at doses higher than 10 mg/kg could only produce 40% inhibition of PBQ-induced writhing (Formukong, Evans, & Evans, 1988). And as mentioned earlier, CBD was the most effective of the cannabinoids, according to Formukong et al. (1988), at doses of 100 mg/kg. Doses of cannabinoids that were effective in the analgesic test orally were also used topically to antagonize TPA-induced erythema of skin.

According to Robson (2001), a number of human studies show that THC is significantly superior to placebo and produces dose-related analgesia peaking at around 5 hours, comparable to but outlasting that of codeine. Side-effects, according to Robson (2001), were also dose-related, and consisted of slurred speech, sedation and mental clouding, blurred vision, dizziness and ataxia. Levonantradol, a THC analog, was also superior to placebo and notably long-acting, but almost half the patients reported sedation. According to the Institute of Medicine (1999), cannabinoids may have considerable potential in treating neuropathic pain.

Noyes et al., (1975) have conducted a double-blind placebo-controlled study on 10 cancer pain patients, administering 5, 10, 15, and 20 mg of THC. They observed pain relief significantly superior to placebo at doses of 15 and 20 mg. In another double-blind placebo-controlled study, Noyes et al., (1975) have administered to 36 cancer pain patients 20 mg of THC and 120 mg of codeine. They observed that codeine and THC were equally effective, but higher dose of THC sedated most patients, and some found its psychoactive effects uncomfortable. In another double-blind placebo-controlled study, Jain et al., (1981) have administered to 56 patients with postoperative pain 1.5, 2, 2.5, 3 mg intermuscular levonantradol. They observed that all doses were significantly superior to placebo (at least P<0.05), but there was no dose-response. 57% of patients reported at least one side-effect, but general acceptability was good, according to Jain et al. (1981). In another double-blind placebo-controlled study, Maurer et al., (1990) have administered to a patient with spinal cord injury 5 mg of THC and 50 mg of codeine. They observed that Δ9-THC and codeine both had an analgesic effect in comparison with placebo. Only THC, however, showed significant effect on spasticity (Maurer, Henn, & Dittrich, 1990). In another double-blind placebo-controlled study, Holdcroft et al., (1997) have administered to a patient with gastro-intestinal (GI) tract pain (familial Mediterranean fever) 50 mg of THC daily. They observed that morphine requirement was significantly reduced (P<0.01) during the active treatment (Holdcroft, Smith, & Jacklin, 1997).

Another constituent of the proposed formulations, in one embodiment, is Mitragyna speciosa plant extract. In addition to analgesic effects, methanolic Mitragyna speciosa extract may demonstrate anti-inflammatory effects. Inflammation is a response to pathogens, chemical or mechanical injury, or based on neurogenic loops (neurogenic inflammation). According to Shaik Mossadeq et al. (2009), an intraperitoneal administration of an Mitragyna speciosa plant extract was able to inhibit the development of a carrageenan induced paw oedema with a maximal inhibition during first 3 hours after the challenge. The extract may exert its anti-inflammatory effect by inhibiting the synthesis, release and action of a number of hyperalgesic mediators. Thereby, it suppresses the early phase of the oedema, which is the characteristic of acute inflammation. Arachidonic acid and its metabolites according to Shaik Mossadeq et al. (2009), might be responsible for the inhibitory activity of the extract for a period of 4 hours. Daily administration of the Mitragyna speciosa plant extract, according to Shaik Mossadeq et al., (2009) was also able to inhibit the growth of granuloma tissue as characterized by proliferation of modified macrophages, fibroblasts and highly vascularized and reddened mass tissue. The authors suggested that inhibition of pro-inflammatory mediator release and vascular permeability in combination with enhanced immunity, stimulation of tissue repair and healing processes may have contributed to the anti-inflammatory properties of Mitragyna speciosa (Shaik Mossadeq, et al., 2009).

Further, according to Kumarnsit et al., (2006) acute and chronically treated rats with Mitragyna speciosa plant extract showed a suppression of food and water intake. Also, weight gain was reduced. In a cellular model in rat L8 myotubes, however, according to Purintrapiban et al., (2011) it was shown that Mitragyna speciose preparations increase the rate of glucose uptake and protein levels of glucose transporters, which may contribute to anti-diabetic effects. Central administration of mitragynine into the lateral ventricle did not alter the basal gastric acid secretion, but administration into fourth ventricle of anesthetized rats caused an inhibition of 2-deoxy-D-glucose-stimulated gastric acid secretion in a dose dependent manner. This inhibition was reversed by naloxone indicating the involvement of opioid receptors. The effects of mitragynine, particularly anorexia and weight loss, might be related to direct inhibition of neurons in the lateral hypothalamus (Tsuchiya, et al., 2002). Subcutaneous 7-hydroxymitragynine also caused an inhibition of the gastrointestinal transit in mice (Matsumoto, et al., 2006).

Kratom is known to produce other effects, some of them, as this invention proposes, may be therapeutic. According to Harizal et al., (2010) acute oral administration of 100, 500 and 1000 mg/kg doses of standardized Mitragyna speciosa methanolic extract increased blood pressure in rats 1 hour after administration. Chittrakarn et al., (2010) as already mentioned, reported that a methanolic Kratom extract caused muscle relaxation in rats. Thereby, the extract had a greater effect at the neuromuscular junction than on the skeletal muscle or at the somatic nerve. According to Chittrakarn et al., (2010) the Kratom extract and mitragynine (2 mg/mL) blocked the nerve conduction, amplitude and duration of compound nerve action potential. In addition to the above reviewed effects, Mitragyna speciosa extract may interact with other drugs changing their metabolism (Hassana, et al., 2013) and effects, as proposed in this invention.

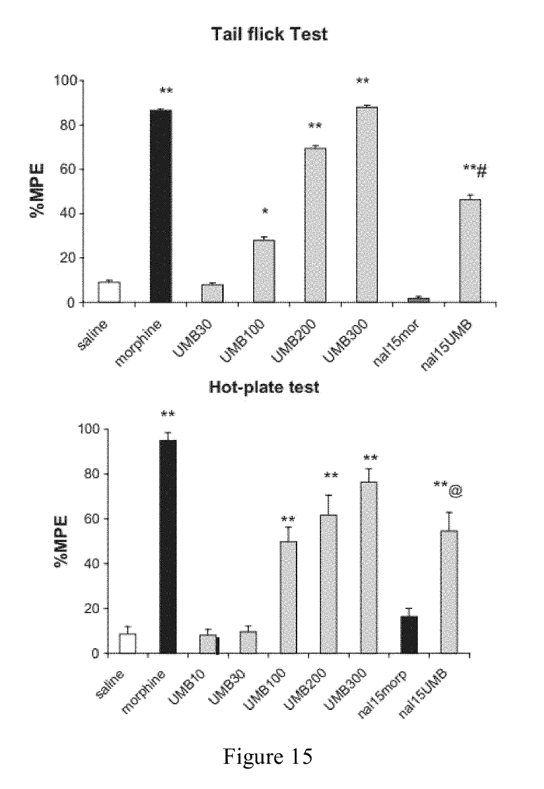

Likewise, Psychotria genus is well-researched and many therapeutic formulations were developed from plants of this genus. Multiple studies have confirmed morphine-comparable analgesic actions of certain Psychotria derived inodole and pyrrolidinoindoline alkaloids. One of the well-known alkaloids derived is hodgkinsine that besides the analgesic activity, exhibits anti-viral, antibacterial, antifungal, and anti-yeast activities, as well as has low cytotoxicity to both dividing and non-dividing cells (Jannic, et al., 1999). In the genus Psychotria, the analgesic activity was identified in alkaloids with diverse molecular structures, such as the indole monoterpene alkaloid umbellatine, and the pyrrolidinoindoline-type alkaloids hodgkinsine and psychotridine. The FIG. 15 shows results obtained by tail-flick model with the alkaloid umbellatine, derived from Psychotria umbellata (Both, Kerber, Henriques, & Elisabetsky, 2002). Umbellatine was clearly active at 100 mg/kg. The analgesic activities of 200 and 300 mg/kg of umbellatine were comparable in efficacy to those of 6 mg/kg of morphine, and the effects were partially reversed by naloxone. The hot plate results are provided in FIG. 15 (Both, Kerber, Henriques, & Elisabetsky, 2002). Umbellatine was dose-dependently active, and its activity was diminished but not completely reversed by naloxone. As with the tail-flick model, 200 and 300 mg/kg activity was comparable in efficacy to that obtained with 6 mg/kg of morphine (Both, Kerber, Henriques, & Elisabetsky, 2002).

Another study conducted by Amador et al., (2001) reports that hodgkinsine, an alkaloid derived from flowers of Psychotria colorata, possesses strong analgesic properties. The study results demonstrate that hodgkinsine produces a dose-dependent naloxone reversible analgesic effect in thermal models of nociception, suggesting that activation of opioid receptors participates in the mode of action of hodgkinsine. In the tail-flick model, the activity of 0.5 mg/kg hodgkinsine is comparable to that of 6.0 mg/kg of morphine. In the hot-plate model, the activity of 20.0 mg/kg of hodgkinsine is comparable with 6.0 mg/kg of morphine (Amador T. A., Verotta, Nunes, & Elisabetsky, 2001).

Going back to Picralima nitida, as in all botanicals, its composition of chemicals somewhat varies from geographical location and from month to month at different harvest times. Phytochemical screening of Picralima nitida has revealed the presence of alkaloids, tannins, saponins, flavonoids, terpenoids, steroids and glycosides in the plant (Erharuyi, Falodun, & Langer, 2014). The seed extract of Picralima nitida, is amber granular powder, with a tendency to clump; bitter, with a characteristic aroma; very soluble in water, and yield a slightly cloudy solution (pH of 5% solution is 5) (Ameh, et al., 2010). The metallic content of Picrafima nitida extract was reported as follows: 2.2 Na, 2.3 K, 6.1 Mg, 41Ca, 0.71 Cr, 3.8 Mn, 16 Fe, 1.9 Zn, 0.77 Ni, 0.33 Cu, 0.07 Pb (Ameh, et al., 2010). Phytochemical investigation has led to the isolation of tannins, saponins, glycosides, alkaloids, flavonoids, steroids, terpenoids, coumestan glycosides derivatives and other phytochemicals. Alkaloids are the major class of phytochemicals isolated from Picralima nitida. The first set of alkaloids isolated are the indole alkaloids: akuammine (also known as vincamajoridine) (C₂₂H₂₆N₂O₄), pseudoakuammine, akuammidine (Rhazine) (C₂₁H₂₄N₂O₃), akuammicine (C₂₀H₂₂N₂O₂), pseudoakuammicine (C₂₀H₂₂N₂O₂), akuammigine (C₂₁H₂₄N₂O₃), pseudoakuammigine (C₂₂H₂₆N₂O₃), picraline (C₂₃H₂₆N₂O₅), akuammiline (C₂₂H₂₄N₂O₄), akuammenine (C₂₀H₂₂N₂O₄) (Saxton, 2014) and picranitine (C₂₁H₂₄N₂O₅) that is identified more recently (Tane, Tene, & Sterner, 2002). Akuammine is the main alkaloid in the seeds of Picralima nitida and was recognized as a 5-hydroxy-N-methylindoline (slightly soluble in water, cold alcohol; soluble in boiling alcohol, chloroform, acetone). Akuammine is strong sympathomimetic and showed antimalarial, antipyretic, anti-inflammatory and analgesic activities (Okada, Tsuda, Salvadori, & Lazarus, 2012). Independently, vincamajoridine, recently isolated from Vinca major, was similarly characterized as a 5-hydroxy-N-methylindoline of the formula C₂₂H₂₆N₂O₄. The descriptions of the two substances and a direct comparison prove that they are identical (Janet, Le Men, Aghoramurthy, & Robinson, 1955).

To date, several alkaloids have been isolated from Vinca major: reserpinine (C₂₂H₂₆N₂O₄), majoridine (C₂₃H₂₈N₂O₃), vincamajoridine (C₂₂H₂₆N₂O₄), vincamine (C₂₁H₂₆N₂O₃), vincamajine (C₂₂H₂₆N₂O₃), perivincine (C₂₂H₂₈N₂O₄), pubescine (C₂₀H₂₆N₂O₄), vinine (C₁₉H₂₆N₂O₄), vincawajine (C₂₄H₂₈N₂O₅), majorinine (C₂₂H₂₄N₂O₄), 10-methoxyvinorine (C₂₂H₂₄N₂O₃)(Sukhdev & Shamsher, 2016). Majoridine and akuammine are indole alkaloids present in the aerial parts of Vinca major and are known for their astringent, antihaemorrhagic and hypotensive effects and used to treat menorrhagia and leucorrhoea (Wren, 1988). According to Singh et al., (2013) the plant's demonstrated biological activity may be due to the consequence of a specific bioactive molecule or it can be the result of synergistic interactions of bioactive molecules.

Several embodiments of this invention propose potentially safer and more effective compounds of Picralima nitida or Vinca major in combination with certain Psychotria genus-derived inodole-type or pyrrolidinoindoline-type alkaloids and alkaloids of Mitragyna speciose, as well as Cannabis sativa alkaloids that are extracted, purified and combined to provide a pharmaceutical-grade compound for treatment of pain, cough and other symptoms. Another embodiment of this invention describes an effective drug delivery system that allows timed-release of the active ingredients necessary to achieve superior efficacy with minimum side effects than the extracts alone.

As already mentioned, Psychotria genus and Mitragyna speciosa contain certain alkaloids, such as the 7-hydroxymitragynine and mitragynine, in case of Mitragyna speciose, and hodgkinsine, in case of Psychotria, that reportedly exhibit similar or more potent analgesic actions than morphine and having reduced side effects, most importantly, in case of mitragynine, significantly reduced respiratory depression and lesser addiction liability due to the presence of kappa-opioid receptor antagonists (Kruegel, et al., 2016)—relatively analogous in mechanism of action to the drug buprenorphine (Falcon, et al., 2016). Picralima nitida and Vinca major also contain certain opioid receptor agonists and antagonists, as mentioned earlier, working in synergy with other substances they create unique therapeutic qualities.

Multiple receptor targets may be beneficial in treatment of pain, and especially complex pain syndromes, such as the neuropathic pain. But unlike the buprenorphine, in some embodiments, when certain Kratom alkaloids are combined with cannabinoids, such as THC and CBD, and the constituents of Picralima nitida and/or Vinca major, the proposed compound provides vasodilating, antihypertensive, muscle relaxing, immune-stimulating, anti-inflammatory, antipyretic, anti-arrhythmic, antitussive, and mild adrenergic effects. The mild stimulation of mitragynine (in low doses), and THC-CBD agonist-antagonist interaction in CB receptors, help to reduce drowsiness associated with higher doses of opioids and THC. The analgesia onset profiles and different elimination times of Kratom and Picralima nitida and/or Vinca major alkaloids, when combined in certain ratios, provide reliable continuous analgesia over an extended period.

Despite the addiction and other concerns, morphine and its derivatives remain to be the primary medicines to treat severe pain and cough. Cannabis-based medicines are also now being offered for treatment of muscle spasticity, epilepsy, and PTSD. For example, Sativex—a CB extract oral spray containing THC and CBD—known to relieve MS spasticity symptoms (Langford, et al., 2013). Sativex and other CB-based medicines can be used to treat neuropathic pain, nausea associated with cancer chemotherapy, as well as stimulate appetite in HIV patients (Lynch, Cesar-Rittenberg & Hohmann, 2014) (Blake, et al., 2017) (Haney, et al., 2007). However, new compounds are needed that can demonstrate comparable or better analgesia but have superior safety and versatility.

Nevertheless, morphine remains to be the indispensable analgesic for improving patients' quality of life (QOL) in the cases of cancer and other illnesses causing severe pain. Also, codeine—another opioid that is still a gold-standard for treating sever cough. It is available in combination with other medicines and without prescription in many countries. However, morphine has problems of low bioavailability and causing various side effects, such as formation of analgesic resistance and physical or psychological dependence due to continued use, nausea and vomiting, constipation, sleepiness, and most importantly respiratory depression (Benyamin, et al., 2008). Large doses of codeine can have similar to morphine side effects.

With that said, the advent of a potent and more safe analgesic and antitussive, serving as a substitute for morphine and codeine, has long been needed. Besides, the proposed compounds have a number of other important qualities that may warrant development of commercial medicines. The level of analgesic action in compounds designed to treat other conditions can be regulated using different ratios of active ingredients. For instance, inflammation and pain management would require a higher content of Psychotria genus and/or Mitragyna speciose extracts; management of influenza (flu) would require in addition to Picralima nitida and/or Vinca major extracts, a Psychotria genus extract or a purified hodgkinsine alkaloid for day-time therapy, considering its anti-inflammatory and other medicinal qualities, and would require no Mitragyna speciose extract, but said extract can be used for night therapy; management of phytosis or mycosis requires reduced quantity of akuammine, akuammidine, pseudoakuammigine, and other analgesics in the extracts but increased concentration of antibacterial constituents; and so on. Manipulation of alkaloid content in the compound is also used to achieve specific therapeutic goals. Another example of little-known therapeutic synergies achievable in the proposed formulations is demonstrated by the antitussive characteristics of Picralima nitida-derived alkaloids and Psychotria colorata-derived hodgkinsine and psychotridine, which are known to act as non-competitive NMDA receptor antagonists (Amador T. A., Verotta, Nunes, & Elisabetsky, 2001) (Amador T. A., Verotta, Nunes, & Elisabetsky, 2001), similar to dextrorphan, a metabolite of dextromethorphan, one of the most commonly used cough suppressants. The synergetic work of these Picralima nitida and Psychotria colorata-derived alkaloids, achievable in certain ratios, provides an enhanced and longer-acting antitussive effect.

The novel compositions and treatment methods, with several variations, some of which are exemplified herein, exhibit extraordinary therapeutic qualities and have potential for commercialization. The proposed invention has relied on new scientific findings, experiments and anecdotal evidence obtained through this research. The inventors believe that this invention is unique and different from the existing art related to Picralima nitida, Mitragyna speciose, Cannabis sativa, Vinca major, and Psychotria genus compounds, processes, and methods. Some of the existing techniques are briefly outlined below.

The U.S. Pat. No. 5,290,553, referenced herein, discloses the invention that relates to a method of preparing substantially purified alkaloids from seeds, stems, and bark of a plant selected from Picralima nitida, Gongronema latifolia, Dorstenia multiradiata, Cola attiensis, Rothmania withfieldii and Desmodium gangeticum, for use in the treatment of protozoal diseases, comprising: pulverizing said plant; a first solvent, drying the extracted material and re-extracting the dried material with a different solvent; extracting a fresh sample of said plant with boiling water; filtering and concentrating the boiling water solvent extracts under reduced pressure; concentrating the dried extract to a gum and re-extracting said gum with an aqueous acidic HCl solution; filtering the acidic extract and making it alkaline to a pH of about 9 with a concentration NaOH solution; extracting the alkaline solution with dichloromethane; concentrating organic layers of the extracted alkaline solution to dryness under reduced pressure to obtain an alkaloid fraction; and separating the alkaloid fraction by liquid chromatography-mass spectrometry to obtain substantially purified alkaloids for use in treatment of protozoal diseases (U.S. Pat. No. 5,290,553, 1991).

The KR. Pat. No. 101,430,354, referenced herein, discloses the invention that relates to an antioxidant or an anti-inflammatory food composition, cosmetic composition, and pharmaceutical composition containing a psychotria rubra extract as an active ingredient. The composition containing the psychotria rubra extract indicates the antioxidant or anti-inflammatory activity, thus can be used to prepare foods, cosmetics, and medicines which can prevent, improve, and treat a disease caused by an oxidation and an inflammatory reaction (Korea Patent No. 101430354, 2013).

The CN. Pat. No. 104,926,840, referenced herein, discloses the invention that relates to a preparation method and an application of plant extract, particularly a preparation method for a psychotria rubra tablet and an application of the psychotria rubra tablet to tumor resistance. The preparation method comprises the following steps: grinding psychotria rubra leaves, performing ultrasonic extraction by using an alcohol solvent to obtain alcohol extract, extracting the alcohol extract by using ethyl acetate, filtering, performing reduced pressure concentration on filter liquor, mixing with a small amount of silica gel, volatilizing the solvent to dryness, passing through a silica gel column, performing gradient elution by dichloromethane-methanol, collecting eluate, performing separation and purification through a high-speed counter-current chromatography method after concentration, and performing freeze-drying to obtain the psychotria rubra tablet. The psychotria rubra tablet has a tumor-resistant effect; and an intestinal absorption enhancer is added in an oral formula of the psychotria rubra tablet, so that the bioavailability can be improved (China Patent No. 104926840, 2015).

The U.S. Pat. No. 4,853,213, referenced herein, discloses the invention that relates to a method of oral hygiene methods for reducing plaque and for the treatment of periodontal diseases of bacterial etiology by significantly reducing bacterial activity in the oral cavity through the inclusion of about 0.03% to at least about 10% by weight of a dried methanol extract of the perennial herb periwinkle in compositions and applying the compositions to the oral cavity (U.S. Pat. No. 4,853,213, 1986).

The U.S. Pat. No. 5,714,163, referenced herein, discloses the invention that relates to a liposome formulation containing a Vinca alkaloid and an ion in an aqueous phase of the liposome. The liposomes also comprise distearoylphosphatidyl choline, cholestrol and disteraroylphosphatidylglycerol. A method for enhancing the efficacy and tumor targeting properties of liposomal vinca alkaloid formulations containing unilamellar vesicles (U.S. Pat. No. 5,714,163, 1994).

The KR. Pat. No. 20,150,096,176, referenced herein, discloses the invention that relates to a medicinal plant vincamine (vincamine), which improves a scalp condition, having an antibacterial effect in a bideumgyun composition. Winkle extract has almost no cytotoxicity while providing good antimicrobial activity (Korea Patent No. 20150096176, 2014).

The U.S. Pat. No. 6,127,377, referenced herein, discloses the invention that relates to novel halogenated derivatives of the vinblastine and vinorelbine family and their therapeutically acceptable salts. The invention also concerns the application of these compounds in therapy and their method of preparation (U.S. Pat. No. 6,127,377, 1997).

### SUMMARY OF THE INVENTION

The following description presents a simplified view of one or more aspects of the proposed invention. This summary is not an extensive overview of all the contemplated embodiments and implementations. It is intended to neither identify key or critical elements of all features, nor delineate the scope of any or all facets. Its sole purpose is to present some concepts of one or more aspects in a simplified form.

It was discovered, that the use of Picralima nitida extracts with certain ratios of constituents or in combination with Cannabis sativa, and/or Mitragyna speciose, and/or Psychotria species plants, containing substances extracted from the plant material are more effective in the treatment of inflammation, pain, reducing symptoms associated with allergies, upper respiratory tract infection, and other conditions contemplated by this invention. It was also discovered that some unwanted side effects caused by the currently available medications can be reduced or eliminated with methods and formulations disclosed herein.

In one embodiment, the proposed invention includes a Picralima nitida plant extract that contains akuammine, akuammidine, akuammicine, and pseudoakuammigine, as well as it includes Mitragyna speciosa plant extract that contains 7-hydroxymitragynine and mitragynine alkaloids. In another embodiment, Picralima nitida plant extract is combined with Cannabis sativa plant extract, containing THC and CBD alkaloids. The respective ranges of THC and CBD, as well as 7-hydroxymitragynine and mitragynine may vary according to the starting plant material and the extraction methodology used. The plants extracts may be obtained by various extraction techniques and the plant material. Such means include but are not limited to: supercritical or subcritical extraction with CO2, extraction with hot gas, and extraction with solvents.

In another embodiment, the proposed invention includes a Picralima nitida plant extract that contains akuammine and at least one pharmacologically inactive substance, and the extract further contains at least a trace amount of pseudoakuammigine, as well as other alkaloids, which are co-extracted from the plant material. In another embodiment, the proposed invention includes a Picralima nitida plant extract and Psychotria species plant extract that contains indole monoterpene-type alkaloids and/or pyrrolidinoindoline-type alkaloids. Their respective ranges may vary according to the starting plant material and the extraction methodology used.

In another embodiment, the proposed invention includes a synthesis of hodgkinsine, using one of the available techniques, such as the one described by Verotta et al. (2002). In another embodiment, the proposed compound contains synthesized mitragynine and at least one pharmacologically inactive substance, and the compound further contains synthesized or Picralima nitida-extracted pseudoakuammigine, as well as at least a trace amount of one other of plurality of synthesized or Picralima nitida-extracted indole alkaloids. Their respective ranges may vary according to the desired clinical effect or the type of symptom relief required.

In another embodiment, the proposed invention includes a Picralima nitida plant extract and at least a trace amount of at least one of the Mitragyna speciosa plant-extracted alkaloids: paynantheine, 3-isopaynantheine, rhynchophylline, mitraphylline, mitrafoline, mitragynine oxindole, mitraciliatine, isospeciofoline, isorhynchophylline, isopteropodine, isomitrafoline, isomitraphylline, 9-hydroxycorynantheidine, epicatechin, corynoxine, corynoxeine, corynantheidine, ciliaphylline, ajmalicine, tetrahydroalstonine, stipulatine, speciophylline, speciophylline, speciofoline, or any combination thereof, or a natural or synthetic analogue thereof and/or derivatives thereof.

And in another embodiment the Picralima nitida plant extract is combined with at least one of the following CBs: CBGA, CBGAM, CBG), CBGM, CBGVA, CBGV, CBCA, CBC, CBCVA, CBCV, CBDA, CBD, CBDM, CBD-C4, CBDVA, CBDV, CBD-C1, THCA-A, THCA-B, THC, THCA-C4, THC-C4, THCVA, THCV, THCA-C1, THC-C1, delta-7-cis-iso-tetrahydrocannabivarin, Δ8-THCA, Δ8-THC, CBLA, CBL, CBLV, CBEA-A, CBEA-B, CBE, CBN, CBNM, CBN-C4, CBV, CBN-C2, CBN-C1, CBND, CBVD, CBT, 10-ethoxy-9-hydroxy-delta-6a-tetrahydrocannabinol, 8,9-dihydroxy-delta-6a-tetrahydrocannabinol, CBTV, CBTVE, DCBF, CBF, CBCN, CBT, OTHC, cis-THC, OH-iso-HHCV, CBR, triOH-THC, or any combination thereof, or a natural or synthetic analogue thereof, and/or derivatives thereof.

In one embodiment of the proposed invention, the treatment of pain and inflammation associated with arthritis involves giving to a patient in the morning by oral administration one soft-gel capsule of the compound containing a mixture of the following pharmacologically active substances: 60% Picralima nitida plant extract of the total compound by mass, and 20% 7-hydroxymitragynine and/or pseudoindoxyl of the total compound by mass, and 10% THC of the total compound by mass, and 10% CBD of the total compound by mass, and a number of pharmacologically inactive substances, such as lipid carriers and stabilizers. Said capsule is a time-released capsule designed to release said mixture in the small intestine; and in another embodiment, in the stomach. The aforesaid compound and method provide an effective arthritis pain and inflammation management option. In one embodiment, the content of THC may be increased to 20% and CBD decreased to null percent, if the medication to be taken before bedtime. In another embodiment, said compound can be administered to reduce neuropathic pain, and it can be used in combination with baclofen and benzodiazepine to lower neuropathic pain and muscle spasticity.

In another embodiment of the proposed invention, the treatment of acute chronic pain involves giving to a patient every 6 hours by oral administration one soft-gel capsule of the compound containing a mixture of the following active substances: 30% THC of the total compound by mass, 50% 7-hydroxymitragynine and/or pseudoindoxyl of the total compound by mass, and the rest is Picralima nitida plant concentrated extract, and where said compound may contain a trace amount of one or more other of plurality of indole or oxindole alkaloids and/or CBs, and a number of pharmacologically inactive substances, such as preservatives, carriers and stabilizers. Said capsule is an immediate release capsule designed to release said mixture in the small intestine; and in another embodiment, in the stomach. The aforesaid compound and method in some subjects may reduce acute pain, such as post-operative pain, trauma-related pain, or acute migraine-related pain.

In another embodiment of the proposed invention, an athlete's performance enhancing plan includes taking by oral administration 30 minutes before the exercise a nutraceutical supplement, one tablet of the compound containing a mixture of the following active substances: 10% mitragynine of the total compound by mass, and less than 1% 7-hydroxymitragynine and/or pseudoindoxyl of the total compound by mass, and 4% epicatechin of the total compound by mass, but not more than 2 mg per kg of bodyweight, 30% CBD of the total compound by mass, and 30% the Picralima nitida plant extract of the total compound by mass, as well as 25% ascorbic acid (C₆H₈O₆) of the total compound by mass; and where said compound may contain a trace amount of other alkaloids. The aforesaid compound and method in some subjects may increase pain tolerance over a period of 6 hours, increase physical and mental endurance, provide additional energy and elevate mental mood, as well as reduce physical and mental fatigue.

In another embodiment of the proposed invention, the symptomatic treatment of common cold involves giving to a patient 4 times in 24 hours by oral administration a dose of medicament, in one embodiment, a syrup compound containing a liquid mixture that includes 500 mg of Picralima nitida plant extract with akuammine representing less than 0.01% of said extract, as well as sugar, water, preservatives, coloring and flavoring agents. This formulation helps to relief symptoms associated with common cold without the unwanted sedative effects of certain alkaloids.

In another embodiment of the proposed invention, the treatment of neurological pain involves giving to a patient 6 times in 24 hours by oral administration a dose of medicament, in one embodiment, a syrup compound containing a liquid mixture that includes 100 mg of Picralima nitida plant extract with akuammine representing not less than 1% of said extract. This formulation provides neuroprotective and anti-inflammatory effects, also relieving neurological pain. In another embodiment this formulation can treat pain associated with lupus erythematosus, fibromyalgia, arthritis, and migraine.

In another embodiment of the proposed invention, the treatment of chronic cough involves giving to a patient 6 times in 24 hours by oral administration a dose of medicament, in one embodiment, a hard-gelatin capsule, encapsulating 250 mg of Picralima nitida plant extract that includes alkaloids and saponins, all granulated with chitosan malate excipient. And in another embodiment containing 50 mg of mitragynine, and 200 mg of citric acid. And in another embodiment containing 100 mg of hodgkinsine, which is known to acts as a non-competitive NMDA receptor antagonist, similar to dextrorphan, a metabolite of dextromethorphan—all, enhancing the antitussive, expectorant, and bronchodilating effects.

In another embodiment of the proposed invention, the treatment of acid reflux involves giving to a patient 30 minutes before a meal by oral administration a dose of medicament, in one embodiment, a tablet consisting of the compound containing 300 mg of Picralima nitida plant extract with akuammine representing less than 0.01% of said extract, and where said compound contains 200 mg of calcium carbonate, and where the tablet includes disintegrant and other excipients. This formulation helps to reduce acid secretion and increase gastric tissues protection, providing relief of symptoms associated with gastritis, acid overproduction, acid reflux, and peptic ulcers without the unwanted sedative effects of certain alkaloids.

In another embodiment of the proposed invention, the symptomatic treatment of seasonal allergy involves giving to a patient as needed, but not more than 6 times in 24 hours, a tableted medicament consisting of 300 mg of Picralima nitida plant concentrated extract with akuammine representing less than 0.01% of said extract, 50 mg of hodgkinsine, and 250 mg of pseudoephedrine, as well as medically acceptable pressed tablet excipients. In another embodiment, said medicament is provided as an oromucosal (sublingual) spray solution, with doses adjusted for the bioavailability of sublingual delivery, containing 2% chitosan bio-availability enhancer and other excipients. These formulations help to conveniently control common allergy symptoms with minimum side effects.

In another embodiment of the proposed invention, the symptomatic treatment of Complex Regional Pain Syndrome involves applying to a patient a transdermal patch that delivers the following dosage of medicament in 24 hours, consisting of 25 mg of akuammine, 10 mg of CBD, 5 mg of THC, 25 mg of 7-hydroxymitragynine and a pharmaceutical-grade ethanol-based vesicular transdermal carrier and other excipients. In another embodiment, the compound includes 50 mg of hodgkinsine or 40 mg of umbellatine. In another embodiment, an ointment or a cream preparation is used to deliver said active ingredients. In another embodiment, said transdermal patch preparation includes another pain relief medicine, such as diclofenac. And in another embodiment said topical cream preparation contains 30% of methyl salicylate. These treatment methods and delivery systems, in case of the transdermal patch, provide sustained release of medicine for up to 7 days, and in case of the ointment and cream formulations, a convenient way to treat pain of peripheral and central generation. Said compounds provide a potent poly-analgesic action working on multiple receptor systems, such as endocannabinoid receptors, opioid receptors, and NMDA ionotropic glutamate receptors.

In another embodiment of the proposed invention, the symptomatic treatment of seasonal allergy involves giving to a patient 6 times in 24 hours a dose of the following medicament as oromucosal (sublingual) spray solution, consisting of 400 mg of Picralima nitida plant aqueous concentrated extract with akuammine representing less than 0.01% of said extract, 2% chitosan bio-availability enhancer and other excipients. This formulation helps to control common allergy symptoms with minimal side effects. In another embodiment, the proposed formulation without the bio-availability enhancer can be used to treat Candidiasis either as an oral spray for oral Candidiasis or a mouthwash, or a spray to treat mucous membranes.

In another embodiment of the proposed invention, the treatment of Tinea corporis involves a topical lotion formulation applied on the affected area 2 times in 24 hours, consisting of 500 mg of Picralima nitida plant concentrated aqueous extract with akuammine representing less than 0.01% of said extract, and 200 mg of Psychotria colorata plant extract with less than 0.1% of hodgkinsine, as well as an emulsifier and other excipients. This dose is approximately sufficient to treat 20 square centimeters of the affected skin. In another embodiment, said medicament is delivered as an aqueous aerosol spray.

In another embodiment of the proposed invention, an athlete after an exercise is given a nutraceutical supplement that is a 355 ml carbonated drink that includes a mixture of 50 mg of Mitragyna speciose plant extract, 10 mg of Cannabis sativa plant extract, and 50 mg of Psychotria colorata plant extract, as well as ascorbic acid (C₆H₈O₆), vitamin B complex, sugar or sugar substitute, preservatives, colorants, flavoring agents, and other ingredients. In another embodiment such nutraceutical supplement is a hard candy; and in another embodiment, it is an energy bar; and in another, a cereal; and in another, a sport nutrient mix.

The proposed invention provides methods and compounds for treatment of multiple diseases and disorders at various stages, and different patients potentially presenting different symptoms, and as such may require larger or smaller doses to achieve the desired efficacy. Besides, the different ratios of active ingredients, other ingredients are required to achieve the desired effect, such as proper absorption.

In one aspect of the invention, titration of doses is beneficial to patients as they can take smaller doses of the medication to achieve efficacy. It is understandable that not all patients will require the same dose of medication, for example, patients of a larger build or faster metabolism may require a higher dose than that required by a patient that is of a smaller build or slower metabolism. In one embodiment said titration is adjusted with a time-release and point of release-tailored dosage forms. For instance, a soft-gelatin capsule designed to release medication in doses in certain parts of the digestive system to achieve the desired efficacy.

In another embodiment, the dose of medicament to be administered to a subject suffering from chronic pain is formulated such that a specific patient can titrate such dose and not develop significant tolerance to the medication; where the term “titrate” means that the patient is provided with a medication that is in such a form or engineered in such a way that smaller doses than the unit dose can be taken. In one embodiment, the titratable dosage forms are gel, gel spray, transdermal patch, liquid, vapor, and the like.

The unit dosage—defined as a maximum dose of medication that can be taken at any one time or within a specified dosage period—may range, in one embodiment, from 5 mg to several grams of medicine, for a patient that just starts using it or was using it continuously for more than 12 months. Depending on the administration route and aforesaid variables, the dosage may fluctuate significantly, such that a unit dose may consist of multiple doses taken several times a day, especially for long-term use patients that have developed tolerance. Administration of the compound may be carried out by any of several suitable known means, including but not limited to intraperitoneal, subcutaneous, oral, intramuscular, intravenous, and other administration forms.

These and other embodiments and objects of the invention will become apparent upon further review of the specification and claims presented herein. Thus, the above and the following expressed embodiments and objects of the invention are not intended by the inventors to limit the scope of the invention.

### BRIEF DESCRIPTION OF THE DRAWINGS

The accompanying drawings, which are incorporated in and constitute a part of this specification, illustrate aspects of the present teachings and together with the description, serve to explain principles of the present teachings.

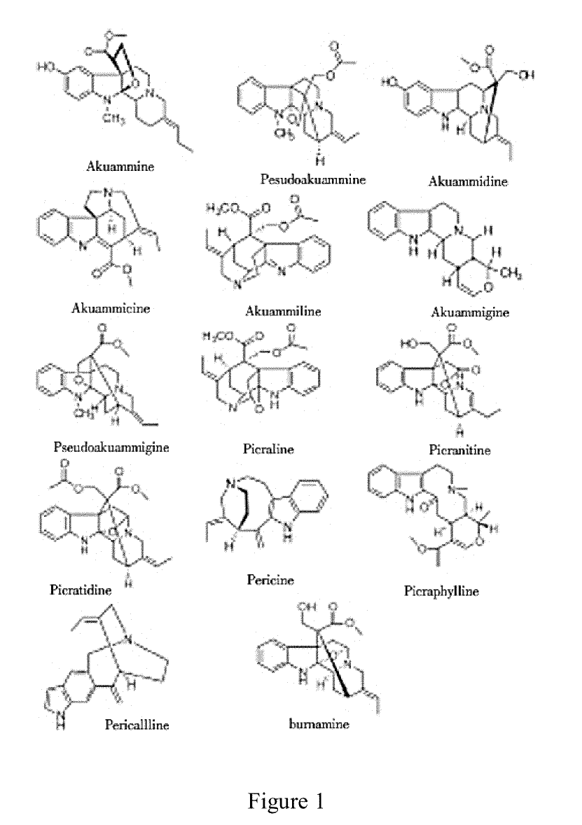

The FIG. 1, incorporated herein by reference, shows chemical structures of alkaloids isolated from Picralima nitida (Erharuyi, Falodun, & Langer, 2014).

The FIG. 2, incorporated herein by reference, provides one example of effects of alkaloids extracted from Picralima nitida on the electrically-evoked contractions of the guinea pig myenteric plexus—longitudinal muscle preparation (upper chart) and of the mouse isolated vas deferens (lower chart) (Menzies, Paterson, Duwiejua, & Corbett, 1998).

The FIG. 3, incorporated herein by reference, shows the affinities of three opioid receptor subtypes of the following compounds: akuammine, pseudoakuammigine (pseudoakuammigine or 10-deoxyakuammine) and (−)-eseroline (Lewin, Le Ménez, Rolland, Renouard, & Giesen-Crouse, 1992).

The FIG. 4, incorporated herein by reference, shows the effect of the extract, diclofenac (D), morphine (M) and normal saline (NS) on the time course curve a, c and e; of the tail immersion test and b, d and f, the AUC in rats. Values plotted are means ±SEM; (n=5). ****P≤0.0001, ***P≤0.001, **P≤0.01, *P≤0.05 compared to vehicle-treated group, (ANOVA followed by Dunnett's post-hoc test) (Dapaah, Koffuor, Mante, & Ben, 2016).

The FIG. 5, incorporated herein by reference, shows the effect of the extract, diclofenac (D) and normal saline (NS), a and c; on the time course curve of acetic acid-induced abdominal writhes; and b and d; on the total nociceptive score in the mice. Data are expressed as mean ±SEM, (n=5). ****P≤0.0001, ***P≤0.001, **P≤0.01, *P≤0.05 compared to vehicle-treated group, (ANOVA followed by Dunnett's post-hoc test) (Dapaah, Koffuor, Mante, & Ben, 2016).

The FIG. 6, incorporated herein by reference, shows the effect of pseudoakuammigine (1.0 (▴), 5.0 (Δ) and 50 (♦) mg kg⁻¹, p.o.; vehicle-treated control (●) and indomethacin (O) 2.5 mg kg⁻¹ p.o.) on: (A) The time-course of carrageenan-induced rat paw oedema and (B) The total oedema response attained during 6 hours. Drugs were administered 1 hour prior to induction of oedema. *=significant (P<0.05) vs vehicle-treated control (Duwiejua, Woode, & Obiri, 2002).

The FIG. 7, incorporated herein by reference, shows the time-course of analgesic effect of pseudoakuammigine (5.0 mg kg⁻¹ p.o.; Δ), morphine (1.0 mg kg⁻¹ s.c.; ▪) and indomethacin (2.5 mg kg⁻¹ p.o.; O) (Duwiejua, Woode, & Obiri, 2002).

The FIG. 8, incorporated herein by reference, shows the potency of pseudoakuammigine (Δ) relative to that of morphine (●) and indomethacin (O) (Duwiejua, Woode, & Obiri, 2002).

The FIG. 9, incorporated herein by reference, shows the effects of Picralima nitida extract, dihydrocodeine (DHC), and normal saline (NS) on the time course curve of (upper left); percent of reduction in cough count and (lower left); percent of increase in latency to cough and (upper right) and (lower right); their AUC's respectively in the citric acid-induced cough test. Data plotted are means ±SEM; (n=5). ****P≤0.0001, ***P≤0.001, **P≤0.01, *P≤0.05, compared to vehicle-treated group (ANOVA followed by Dunnett'spost-hoc test) (Dapaah, Koffuor, Mante, & Ben, 2016).

The FIG. 10, incorporated herein by reference, shows on the upper chart the effect of Picralima nitida extract, atropine (ATR) and normal saline (NS) on acetylcholine-induced bronchoconstriction. Values plotted are means ±SEM; (n=5). ****P≤0.0001, ***P≤0.001, *P≤0.05, compared to vehicle-treated group (ANOVA followed by Dunnett's post-hoc test). And on the lower chart it shows the effect of Picralima nitida extract, mepyramine (MEP) and normal saline (NS) on bronchospasm induced by histamine. Values plotted are means ±SEM; (n=5). ****P≤0.0001, **P≤0.01 compared to vehicle-treated group (ANOVA followed by Dunnett's post-hoc test) (Dapaah, Koffuor, Mante, & Ben, 2016).

The FIG. 11, incorporated herein by reference, shows (the upper chart) the effects of Picralima nitida extract, ammonium chloride (NH₄Cl), and normal saline (NS) on tracheal phenol red secretion in mice as a measure of the expectorant effect. Values plotted are means ±SEM; (n=5). **P≤0.01, *P≤0.05 compared to vehicle-treated group, (ANOVA followed by Dunnett's post-hoc test) (Dapaah, Koffuor, Mante, & Ben, 2016). And on the lower chart it shows the effect of Picralima nitida extract (100, 300, 500 mg/kg), sodium cromoglycate (100 mg/kg), and normal saline on ammonium chloride-induced tracheal phenol red secretion as a measure of muco-suppressant effect. Values plotted are means ±SEM of n=5. Ns implies P>0.05; ***P≤0.001; **P≤0.01; *P≤0.05 compared to vehicle-treated group; (ANOVA followed by Dunnett's post-hoc test) (Dapaah, Koffuor, Mante, & Ben, 2016).

The FIG. 12, incorporated herein by reference, shows (the upper chart) the effects of Picralima nitida extract (100, 250, 500 μg/ml), Ketotifen (10 μg/ml), and normal saline on mast cell degranulation induced by Compound 48/80. Values plotted are means ±SEM, (n=3).ns implies P>0.05; ****P≤0.0001;***P≤0.001; **P≤0.01 compared to vehicle-treated group; (ANOVA followed by Dunnett's post-hoc test) (Dapaah, Koffuor, Mante, & Ben, 2016). And on the lower chart it shows the effect of Picralima nitida seed extract (PNE) free radical scavenging ability of PNE (0.01-0.3 mg/ml) compared to ascorbic acid (0.01-0.3mg/ml) in the DPPH radical assay. Values plotted are means+SEM, n=3 (Dapaah, Koffuor, Mante, & Ben, 2016).

The FIG. 13, incorporated herein by reference, shows the anxiolytic effects of Picralima nitida seed extract (100, 300, 500 mg/kg) compared with said effects of caffeine (10, 30, 100 mg/kg) and diazepam (0.1, 0.3, 1 mg/kg) on the number of arm entries and the % number of arm entries in the elevated plus maze. Data are presented as group mean±SEM (n=6). Significantly different from control: *P<0.05, **P<0.01, ***P<0.001, ****P<0.0001. Also, a diverse significant difference is observed when the zonal entries were compared to each other (*P<0.05). (Two-way repeated measures ANOVA followed by Bonferroni's post hoc test) (Dapaah, Koffuor, Mante, & Ben, 2016).

The FIG. 14, incorporated herein by reference, shows the antibacterial activity of the leaf extract of Picralima nitida (upper table). The upper table figures are in millimeters and include the diameter of the paper disc (5 mm). Data are means of triplicate determinations and MIC is Minimum Inhibitory Concentration (Igwe & Mgbemena, 2014). The figure further depicts antibacterial activity of Picralima nitida seed extract (middle table). The values quoted as zones of inhibitions mean ±SEM; n=3. (−) indicates “no zones of inhibition were observed”. Diameter of borer: 11 mm. ns P>0.05, *P≤0.05, **P<0.01, ***P<0.001, ****P<0.0001 (ANOVA followed by Bonferroni's post hoc test) (Dapaah, Koffuor, Mante, & Ben, 2016). The lower table depicts observations recorded showing inhibitory effect of the Picralima nitida leaf extract on funguses A. flavus, C. Albicans and M. canis (Ubulom, Imandeh, Udobi, & 2012). Values are expressed as mean +SEM (n=3). Positive control=ketoconazole (30 mg/ml); Negative control=test organism, minus extract solution.

The FIG. 15, incorporated herein by reference, shows the effects of Psychotria umbellate-derived umbellatine in the tail-flick (upper chart) and hot plate (lower chart) models. UMB=umbellatine (10-300 mg/kg); nalmorp=naloxone 15 mg/ kg+morphine; nalUMB=naloxone 15 mg/kg+umbellatine (200 mg/kg (upper chart); 300 mg/kg (lower chart)). n=6-8. Columns represent % of Maximum Possible Effect (% MPE) and vertical bars±SD.*=p<0.05, and **=p<0.01 compared to saline; #=p<0.01 compared to umbellatine 200 mg/kg; @=p<0.01×umbellatine 300 mg/kg ANOVA/SNK (Both, Kerber, Henriques, & Elisabetsky, 2002).

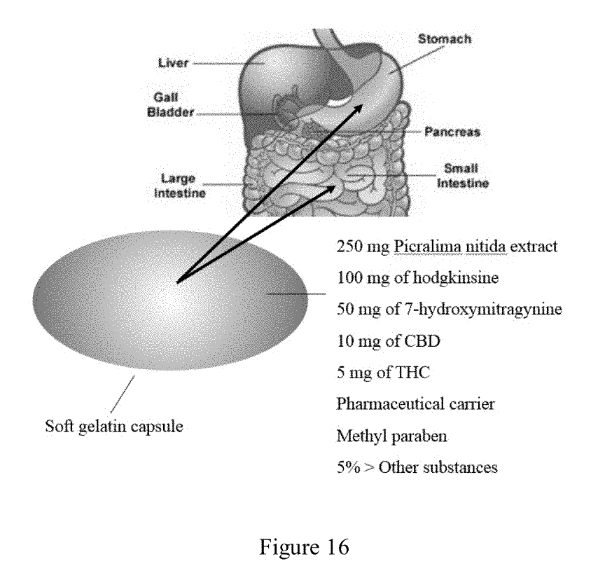

The FIG. 16, incorporated herein by reference, shows a variable-release soft-gelatin capsule pill, one of many possible dosage forms, that consists of predominantly type A or B gelatin, water, sorbitol, and encapsulates a compound containing a liquid mixture that includes: 250 mg of Picralima nitida plant concentrated extract, 100 mg of hodgkinsine, 50 mg of 7-hydroxymitragynine, 10 mg of CBD, 5 mg of THC, a pharmaceutically acceptable carrier, methyl paraben, and less than 5% of other alkaloids and other substances.

### DESCRIPTION OF EMBODIMENTS

Reference will now be made to embodiments, examples of which are illustrated in the accompanying material. In the following description, some details are set forth in order to provide understanding of the proposed invention. However, it will be apparent to one of ordinary skill in the art that the present invention may be practiced without these details. In other instances, well-known methods, procedures, components, circuits, and networks have not been described in detail so as not to unnecessarily obscure aspects of the embodiments.

It will also be understood that, although the terms first, second, etc., may be used herein to describe various elements, these elements should not be limited by these terms. These terms are only used to distinguish one element from another. For example, a first contact could be termed a second contact, and, similarly, a second contact could be termed a first contact, without departing from the scope of the present invention. The first contact and the second contact are both contacts, but they are not the same contact.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. Moreover, the term “or” is intended to mean an inclusive “or” rather than an exclusive “or.” That is, unless specified otherwise, or clear from the context, the phrase “X employs A or B” is intended to mean any of the natural inclusive permutations. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if is detected” may be construed to mean “upon determining” or “in response to determining” or “upon detecting (the stated condition or event)” or “in response to detecting (the stated condition or event),” depending on the context.

As used herein, the terms “related”, “in connection”, or “associated”, or “relevant”, and similar, depending on the context, means any association, whether direct or indirect, by any applicable criteria as the case may be.

The word “exemplary” is used herein to mean “serving as an example, instance, or illustration”. And no aspect of this disclosure shall be construed as preferred or advantageous over other aspects or designs unless expressly stated.

The term “cannabinoid(s)” represents cannabinoid receptor agonists and a group of C21 terpenophenolic compounds found in Cannabis sativa L, as well as synthetic or semisynthetic cannabinoids, for instance, without limitation: nabilone, dexanabinol, ajulemic acid; and cannabinoid receptor ligands that are chemically different endocannabinoids, for instance, without limitation: anandamide; 2-arachidonoylglycerol; and other phytocannabinoids; levonantradol; CP 47,497; (C6)-CP 47,497; (C8)-CP 47,497; (C9)-CP 47,497; CP 50,556-1; CP 55,244; CP 55,940; CP-945,598; HHC; O-1871; AMG-36; AMG-41; AM-694; AM-906; AM-1235; AM-2232; AM-2233; AM-2389 O-1812; THJ-2201; JWH-018 and others.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkali metal salts, sodium, potassium or lithium.

As used herein, “treating” means preventing, slowing, halting, or reversing the progression of a disease or infection. Treating may also mean improving or attempting to improve one or more symptoms of a disease or infection

As used herein, “trace amount” means as practiced in analytical chemistry—average concentration is less than 100 parts per million (ppm) measured in atomic count or less than 100 micrograms per gram.

As used herein, “extract” in reference of amounts or proportions is contemplated to be a dried organic layer in solid state with solvents and water not present beyond a trace amount unless explicitly specified otherwise.

As used herein, “a pharmacologically inactive substance” means a chemical substance which does not significantly increase or affect the therapeutic action of the active ingredient.

The term “subject” or “patient” refers to a mammal in need of treatment or undergoing treatment using the inventive compounds described herein. Mammalian subjects include without limitation humans, dog, cat, horse or any other animal in need of treatment.

As used herein, the percent by mass of a mixture is obtained by dividing the mass of each component by the total mass and multiply by 100 (Percent by mass=mass of component/total mass×100%). For example, a mixture that contains 1.203 g CaCO₃ and 1.797 g NaCl is equal to CaCO₃=40.10% and NaCl=59.90%.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

The present invention provides a number of pharmaceutical compounds that represent a stable, fast-acting formulations of naturally occurring substances or their analogs (for the purpose of this document, may be used interchangeably). An analog herein refers to a compound that is derived by chemical, biological, genetic or synthetic transformation of the naturally occurring substances of Picralima nitida, Mitragyna speciose, Cannabis sativa and Psychotria species plants. The plant extract, referred herein, unless otherwise specified, means an extract from any part of the plant.

The natural alkaloid and other substances are readily obtained from plant tissue by suspending the tissue in an appropriate solvent to extract alkaloid compounds and other tissue components. Analytical purification of such an extract provides pharmaceutical grade alkaloid compounds and other substances. It was reported, for example, that the most abundant opioid alkaloid in the crude extract of Picralima nitida is akuammine at 0.56% of dry mass of the total crude extract, which was isolated and partially characterized in the 1920s (Menzies, Paterson, Duwiejua, & Corbett, 1998). Akuammidine and akuammigine are found in much smaller amounts, 0.034% and 0.01% dry mass of total crude extract, respectively. And akuammicine with pseudoakuammigine are found at 0.006% dry mass of the total crude extract.

There are many reports and techniques of Picralima nitida extraction and its constituents' isolation. Menzies et al., (1998) reports that dried and powdered seeds were treated first with n-hexane (extracting mainly terpenoids and some alkaloids), then with ethyl acetate. The ethyl acetate fractions were applied to alumina columns (Al₂O₃, Spence type H) from which the alkaloids were isolated by elution in n-hexane:ethyl acetate (1:1). The identities and purities of the dried, isolated alkaloids were confirmed by H-Nuclear Magnetic Resonance spectroscopy. Using this method each alkaloid sample was shown to have purity 98% (Menzies, Paterson, Duwiejua, & Corbett, 1998).

Koffi et al., (2014) reports preparation of plant extract in the laboratory, where one thousand grams of the fresh seeds of Picralima nitida were rinsed then introduced in 4 liters of distilled water. The mixture was boiled for 45 minutes, then it was wrung in a cloth square, filtered successively twice on absorbent cotton and on Wattman 3 mm paper. The volume of the filtrate obtained was concentrated with Rotavapor and evaporated in a drying oven at 60° C. for 2 days. The pulverized crystals made it possible to obtain a fine powder (41 g) used for the experimentation (Koffi, Emma, & Stephan, 2014).

Another extraction performed by Igwe et al., (2014) involved extracting with 2 liters of ethanol 3 times for 8 hours at 30° C. 500 g of the powdered Picralima nitida plant leaves. The extract was concentrated under reduced pressure and the supernatant leaf (7.35 g) extract was decanted after complete removal of the solvent. The extract was centrifuged at 10,000 rpm for 20 minutes and the clear supernatant extract was subjected to systematic GC-MS analysis. The components of the ethanolic extract of leaves were identified by matching the peaks with Wiley MS libraries and they were confirmed by comparing mass spectra of the peaks and those from the literature (Igwe & Mgbemena, 2014).

Another extraction and purification were performed by Tane et al., (2002) where dried powdered seeds of Picralima nitida (2.0 kg) were extracted sequentially with ethanol, and the extract obtained was concentrated under vacuum to yield 97 g of residue. This extract was triturated with 0.1 N HCl and the combined acidic solution was exhaustively partitioned with CH₂Cl₂ to give 17 g of CH₂Cl₂ extract. The aqueous layer was adjusted to pH 9 with a solution of 10% NH₃ and the precipitate was filtered to obtain alkaloids that were dissolved in CH₂Cl₂ and washed several times with water. The organic layer was dried with Na₂SO₄ and concentrated to give 54 g of alkaloid. It was then subjected to chromatographic separation over a column of Al₂O₃. Elution was performed with a mixture of hexane-ethyl acetate with increasing polarity. Fractions of 250 mL were collected and monitored by TLC with appropriate solvent system to give three main portions. The portion eluted with hexane/ethyl acetate (7/3) contained a mixture of picratidine and pseudoakuammine purified by a second column chromatography followed by recrystallization. Akuammicine, which was the main constituent of fractions collected with hexane/ethyl acetate (6/4), was purified by recrystallization to give 33 mg of the product. Portions collected with 50% and 70% of ethyl acetate in hexane contained picranitine, akuammine and akuammidine were regrouped and subjected to column chromatography and pure product obtained by fractional recrystallization to yield 60 mg of picranitine, 23 mg of akuammine and 70 mg of akuammidine (Tane, Tene, & Sterner, 2002).

Another extraction and purification was performed by Ubulom et al., (2012) where Picralima nitida leaves were first air-dried on laboratory tables at room temperature (28+2° C.). This was followed by pulverization using the crusher machine, where 500 g of the pulverized leaves were macerated separately in distilled water and 50% ethanol for 72 hours, with periodic stirring. Each extract was filtered repeatedly using muslin cloth, non-absorbent cotton wool and Whatman No. 1 filter paper. This was done to get rid of the marc. The aqueous filtrate was concentrated using a lyophiliser, while the ethanolic filtrate was first concentrated in vacuo at 40° C. using a rotary evaporator, after which it was freeze-dried using the lyophiliser (Ubulom, Imandeh, Udobi, & IIya, 2012).

Another extraction and purification were performed by Erharuyi et al., (2012) where the preparation of extract involved the following: Powdered plant material (3.2 kg) was extracted with 14 liters of methanol by maceration at room temperature for two weeks. The extract was concentrated to dryness using a rotary evaporator at reduced pressure. The concentrated extract was weighed and stored in an air-tight container and kept in the refrigerator at 4° C. The fractionation of extract was performed as follows: The crude methanol extract was subjected to prefractionation/partitioning using different solvents. The crude extract (230 g) was first defatted with 7.5 liters of petroleum ether; the ether insoluble portion was extracted with 16 liters of chloroform followed by 7.5 liters of ethyl acetate. The various fractions were concentrated to dryness, weighed and kept at 4° C. in an air-tight container (Erharuyi & Falodun, 2012).

Another extraction and purification were performed by Dapaah et al., (2016) where the pods of Picralima nitida were opened, and the seeds removed, air-dried, and milled into powder. A 3 kg quantity of the powder was extracted with 70% ethanol by cold maceration over a 72 hours period. The extract obtained was concentrated at 40° C. and under low pressure using a rotary evaporator to a syrupy mass. The syrupy mass obtained was then dried in a hot air oven maintained at 40° C. to obtain 0.389 kg (yield: 12.9%) of a solid mass of Picralima nitida extract (PNE) (Dapaah, Koffuor, Mante, & Ben, 2016).

Phytochemical tests were performed on PNE by Dapaah et al., (2016) to determine the presence of tannins, saponins, glycosides, alkaloids, flavonoids, steroids and terpenoids. The following procedures were used. Glycosides were tested as follows: About 200 mg of PNE was warmed with 5 ml dilute H₂SO₄ on a water bath for 2 minutes. It was then cooled and filtered. The filtrate was made alkaline with 2 to 5 drops of 20% NaOH and 1 ml each of Fehlings solution A and B was then added to the filtrate and heated on the water bath for 2 minutes and observed for the appearance of a brick-red precipitate. Saponins were tested as follows: An amount of 0.2 g of PNE was shaken with few milliliters of water and the mixture observed for the presence of a froth which does not readily break upon standing. Tannins were tested as follows: About 0.5 g of PNE was boiled with 25 ml of water for 5 minutes. It was then cooled, filtered and the volume of the filtrate adjusted to 25 ml with water. To 1 ml of the filtrate, 10 ml of water and 5 drops of 1% lead acetate was added. The color and amount of precipitate, if any, was noted and recorded. The procedure was repeated using 5 drops of 1% ferric chloride. Terpenoids were tested as follows: An amount of 0.5 g of PNE was extracted with 2 ml of chloroform in a test tube followed by addition of 1 ml of concentrated sulfuric acid. The presence of terpenoids was identified by appearance of a reddish-brown coloration at the interface. Steroids were tested as follows: About 0.5 g PNE was extracted with 2 ml of chloroform in a test tube followed by addition of acetic anhydride. Concentrated sulfuric acid was added to the walls of the test tube. Appearance of a blue color at the interface indicates the presence of steroids. Flavonoids were tested as follows: About 0.5 g of PNE was extracted with 2 ml of chloroform in a test tube and 2 ml of methanol added to dissolve it. Concentrated hydrochloric acid was then added together with four pieces of magnesium ribbons. A reddish or pink color indicates the presence of flavonoids. Anthraquinones were tested as follows: About 0.5 g of PNE was each extracted separately with 10 ml of benzene and filtered. About 5 ml of 10% ammonia was added to the filtrate and shaken. A reddish or pink coloration is a positive test for anthraquinones. Alkaloids were tested as follows: About 0.5 g of PNE was boiled with 10 ml of dilute HCl in a test tube for 5 minutes. The supernatant was filtered and 3 drops of Dragendorff s reagent (potassium bismuth iodide solution) added to 1 ml of the filtrate in the test tube. The mixture was then shaken and observed for the appearance of an orange-red precipitate (Dapaah, Koffuor, Mante, & Ben, 2016).

Singh et al., (2017) reports preparation of Vinca major plant extract, where the 5 g of shade dried flowers were grounded, suspended in 100 ml petroleum ether and extracted overnight. Next day, petroleum ether layer was collected and evaporated at 37 ° C. to yield the ether fraction (31 mg; 0.62% percent extractive). Remaining residue was then extracted overnight with 100 ml benzene and fraction was collected by evaporation (34 mg; 0.68% percent extractive). Likewise, chloroform, acetone, methanol and water extracts were also obtained. The process resulted in completely dried 59 mg chloroform extract (1.18% percent extractive), 42 mg acetone extract (0.84% percent extractive), 88 mg methanol extract (1.76% percent extractive) and 107 mg aqueous extract (2.14%) from the Vinca major flowers (Singh, Jarial, & Kanwar, 2013).

Amador et al., (1996) reports a preparation of Psychotria colorata alkaloid extracts, where the dried milled leaves (515 g) or flowers (64 g) were wetted with 6 N NH₄OH and extracted with ethanol in a Soxhlet apparatus. After distillation of solvent, the crude ethanol extract was poured into a 2% acetic acid solution, left for 12 hours in refrigerator and filtered. The clear acidic solution was extracted with chloroform (residue discarded). The aqueous phase was adjusted to pH 7 with NaHCO₃ pH 7.0 and extracted with chloroform; the organic layer washed, dried (Na₂SO₄) and evaporated. TLC analysis showed a very similar composition for both alkaloid extracts. The HPLC/MS analyses identified a mixture of pyrrolidinoindoline alkaloids (e.g., quadrigemine C, calycanthine, isocalycanthine, among others) (Amador, Elisabetsky, & Onofre de Souza, 1996).

Another extraction and purification were performed by Both et al., (2002) where 100 g of dried Psychotria umbellata leaves were extracted with EtOH at room temperature 3-times, each for a week. The extract was concentrated under vacuum at 40° C. to a dark green syrup. The syrup was dissolved in HCl 2% (0.51) and partitioned with CH₂Cl₂. The acid solution was alkalinized with NH₄OH 25% until pH=10 and extracted with CH₂Cl₂. From the CH₂Cl₂ extract 954 mg of a colorless amorphous compound was precipitated. Purity of the compound was checked by TLC with silica gel 60 F254 (CHCl₃MeOH/NH₃ vapor—85:15—RF=0.2) and HPLC (column: NOVAPACK Cis 150 mm×3.9 mm—Waters; MeOH:H₂O—50:50 as eluent and a Photo Diode Array as detector; Rt=2.13 min) (Both, Kerber, Henriques, & Elisabetsky, 2002).

Chittrakarn, et al. (2010), had performed extraction and isolation of Kratom alkaloids from leaves red vein type Kratom. Air-dried leaves were pulverized by grinding and then macerated, at room temperature, with absolute methanol for 7 days, twice, while stirring 2-3 times/day. The extracts were mixed, filtered and concentrated using a rotary evaporator and then they were freeze-dried. The yield was 7.92% (w/w) (Chittrakarn, Keawpradub, Sawangjaroen, Kansenalak, & Benjamas, 2010). According to Chittrakarn, et al., (2010) the isolation of crude alkaloids from the methanolic extract of Kratom leaves was made by dissolving it in 10% acetic acid solution. This solution was shaken and left overnight. The acidic filtrate was washed with petroleum ether, adjusted to pH 9 with 25% ammonia solution, and then extracted with chloroform. The chloroform extract was washed with distilled water, dried over anhydrous sodium sulfate and evaporated to yield a dry crude alkaloid extract. According to the isolation procedure, Chittrakarn, et al., (2010) report that the yield of crude alkaloid extract was approximately 0.25% based on fresh weight of Mitragyna speciosa. An aliquot (2.5 g) was then subjected to silica gel column chromatography, eluted with 5% methanol in chloroform to obtain a major alkaloid (1.25 g), which appeared as a single spot on TLC analysis (four different solvent systems). Over all, the yield of mitragynine in the methanolic extract was approximately 1.56% (Chittrakarn, Keawpradub, Sawangjaroen, Kansenalak, & Benjamas, 2010).

It is preferable, in one embodiment, that the extraction/production method yields substantially the mitragynine and 7-hydroxymitragynine that are believed to be the most effective alkaloids for pain management. There are also various other techniques that are known for extracting and isolating mitragynine and 7-hydroxymitragynine from Mitragyna speciosa plant. For example, Pat. No. CN 102,048,857, describes a method for extracting alkaloids from Kratom (CN Patent No. 102,048,857, 2009).

The natural CB compounds are also readily obtained from plant tissue by suspending the tissue in an appropriate solvent to extract CB compounds and other tissue components. Analytical purification of such an extract provides pharmaceutical grade CB compounds. Alternatively, CB compounds are extracted from plant tissue under supercritical conditions. Solvents used for supercritical extraction of CBs include, for instance: carbon dioxide, or other gases in isolation or combination with or without solvent modifiers, selected from ethanol, propanol, butanol, hexane, chloroform, dichloromethane, acetone, or any organic solvent capable of extracting CBs, and alcohol-water mixtures, such as water-ethanol or water-butanol mixtures, etc.

The present invention, in one embodiment, involves a formulation containing an extract from Cannabis plant matter with THC, CBD and optionally the carboxylic acids thereof. In one embodiment, the dried plant matter is ground and subjected to a CO2 extraction and the primary extract obtained is separated. Specifically, ground Cannabis plant material is compressed and charged into an extraction vessel. CO2 is then introduced, having been brought to a temperature, in one embodiment, of approximately 60° C. and to a pressure of approximately 250 bars. When the CO2 enters into contact with the material to be extracted, it extracts the desired CB components, in particular comprising Δ9-THC and CBD, as well as the carboxylic acids thereof. In one embodiment, the extraction method permits extracting various isomers of THC, selectively obtained from industrial hemp and from drug-producing hemp, also separating undesirable waxes and removing the solvent.

The CBs, including THC, can be isolated from Cannabis plants using extraction methods or can be made synthetically or semi-synthetically. It is preferable, in one embodiment, that the extraction/production method yields substantially the (−)-Δ⁹-trans-THC isomer that is the most active isomer of THC. There are also various techniques that are known for isolating and separating the (−)-Δ⁹-trans-THC isomer from other compounds in THC. For example, U.S. Pat. No. 7,449,589 describes methods for purifying the (−)-Δ⁹-trans-THC isomer from a mixture of other THC isomers (U.S. Pat. No. 7,449,589, 2004).

Similar to Cannabis sativa, substances can be extracted from Picralima nitida, Mitragyna speciose, Vinca major, and Psychotria species plants under supercritical conditions. Solvents used for supercritical extraction of alkaloids and other substances include without limitation: carbon dioxide, or other gases in isolation or combination with or without solvent modifiers, selected from ethanol, propanol, butanol, hexane, chloroform, dichloromethane, acetone, or any organic solvent capable of extracting such substances, and alcohol-water mixtures, for instance, water-ethanol or water-butanol mixtures, and others.

It is not the purpose of this disclosure to provide particulars concerning the attainment of a colloidal formulation that is stable under a range of conditions. However, in one embodiment, the disclosed compound with initial purity (HPLC) of akuammine, 7-hydroxymitragynine, and delta-9-THC being at least 98% by area can achieve stability such that at least 95% by area remains in undegraded form after exposure of the compound to the storage conditions for twelve months, where the ambient temperature is between 20° C. and 40° C. and relative humidity is between 55% and 75%.

In one embodiment, the stability of said compound is attained by contacting a solution containing akuammine, psychollatine, and delta-9-THC into a solvent such as organic solvents, including acetone, acetic acid, alcohols, chloroform, diethyl ether solvents, and other solvents that can be used to dissolve said alkaloids; and in another embodiment, with addition of pharmaceutically acceptable buffers, stabilizers, and other pharmacologically inactive substances.

In one embodiment, the compound of this invention is present in the form of micelles or liposomes that encapsulate mitragynine, 7-hydroxymitragynine, THC, CBD, and/or other alkaloids within the membrane of the micelles or liposomes. Within the context of the present technology, the term “micelle” refers to an aggregate of surfactant molecules dispersed in a liquid colloid, while “liposome” refers to a vesicle composed of a mono or bilayer lipid.

In yet another embodiment, other drugs, and pharmaceutically acceptable carriers, if present, may be in the lipophilic membrane or entrapped in the aqueous fluid that forms the core of the liposome. The entrapped alkaloids contribute to the stability of the micelle/liposome membranes, such that the micelle/liposomes formulations may be used as an improved, fast, reliable and efficient system for the oral, enteral, parenteral, intravenous or topical delivery of mitragynine, pseudoindoxyl, 7-hydroxymitragynine, delta-9-THC, and/or other alkaloids, and/or additional drugs to subjects in need thereof

In another embodiment, unilamellar micelles or liposomes that are thermostable at temperatures greater than 50° C. are used in the manufacture of the compound contemplated by this invention. These micelles or liposomes are obtained by contacting a solution of Mitragyna speciosa and Cannabis sativa plants alkaloids with an appropriate solvent. The mixing of said alkaloid solution occurs in a manner suitable for the rapid dissolution of the alkaloid solution. This can be accomplished through a variety of means including dilution, injection through a small orifice under pressure, and ultrasonic atomization.

And yet in another embodiment, the disclosed compound has advantageous properties, where the micellar and liposomal compound is stable at high temperatures, exceeding 50° C., is stable to sonication, capable of carrying large payloads of Mitragyna speciosa and Cannabis sativa plants alkaloids as well as other drugs suitable for use in combination therapy and can be stored for extended periods of time, for example greater than 20 weeks at 25° C.

In certain embodiments, said compound can be in the form of a concentrated, stable colloidal suspension that is obtained by infusing a solvent solution containing the Psychotria umbellata and Picralima nitida plants extract or essentially pure alkaloids into a solvent, with or without buffer. Stabilizing agent, for instance, a polymer or compounds selected from cellulose hyaluronic acid, citric acid, Tris base, sodium carbonate, polyvinyl pyrrolidone (PVP), alginate, chondritin sulfate, poly gamma glutamic acid, gelatin, chitisin, chitosan, corn starch and flour can be used to stabilize the micelle formulations.

In one embodiment, said compound also exhibits superior systemic delivery and release of Mitragyna speciosa and Cannabis sativa plants alkaloids from the micelle or liposomes used in the manufacture of the contemplated compound. The release of alkaloids from a liposome or micelle of the contemplated compound can be modulated by changing the ratio of the concentration of lipid to the concentration of alkaloids present in the liposome.

In one embodiment, tissue specific delivery can be achieved by modifying the surface of the liposomes or micelles with compounds that bind specifically to biological macromolecules expressed on cellular surfaces. For instance, the micelle or liposomal surface can be derivatized to display an antibody specific to an antigen expressed on the affected cells.

According to one embodiment, said compound that is used in the treatment of a disease condition or other therapy is administered to a patient or subject in need of treatment either alone or in combination with other compounds/drugs having similar or different biological activities. For example, said compound may be administered in a combination therapy, i.e., either simultaneously in single or separate dosage forms or in separate dosage forms within hours or days of each other. Examples of compounds/drugs used in such combination therapies include without limitation: chemotherapeutic agents, immunosuppressive agents, immunostimulatory, anti-pyretic, cytokines, opioids, cannabinoids, cytokines, cytotoxic agents, nucleolytic compounds, radioactive isotopes, receptors, pro-drug activating enzymes, which may be naturally occurring or produced by recombinant methods, anti-inflammatory agents, antibiotics, protease inhibitors, growth factors, osteo-inductive factors and the like.

In some embodiments, the compound further contains, in accordance with accepted practices of pharmaceutical compounding, one or more pharmaceutically acceptable excipients, including without limitation: diluents, adjuvants, stabilizers, emulsifiers, preservatives, colorants, buffers, flavor imparting agents. As stated above, said compounds may contain Cannabis sativa plants alkaloids, their analogs (such as: levonantradol; CP 47,497; (C6)-CP 47,497; (C8)-CP 47,497; (C9)-CP 47,497; CP 50,556-1; CP 55,244; CP 55,940; CP-945,598; HHC; O-1871; AMG-36; AMG-41; AM-694; AM-906; AM-1235; AM-2232; AM-2233; AM-2389 O-1812; THJ-2201; JWH-018 and other cannabinoid receptor agonists), and co-extraction substances, and may be consumed directly or formulated into nutraceutical or pharmaceutically acceptable compounds suitable for oral, enteral, parenteral, intravenous or topical administration.

The term “parenteral” as used herein includes subcutaneous injections, intravenous, intramuscular, intrasternal injection or infusion techniques. Dosage forms for oral administration include food, beverages, drinks, soups, baked goods, syrups, oral pharmaceutical compounds, nutraceutical formulations, and the like. Suitable pharmaceutical carriers include any such materials known in the art, e.g., any liquid, gel, solvent, liquid diluent, solubilizer, polymer or the like, which does not significantly interact with other components of the formulations in a deleterious manner.

Liquid dosage forms for oral administration include, but are not limited to, pharmaceutically acceptable emulsions, solutions, suspensions, syrups and elixirs. In addition to the Cannabis sativa, Picralima nitida, Mitragyna speciose, Vinca major, and Psychotria species plants extracts, the liquid dosage forms can contain inert diluents commonly used in the art. For instance, liquid formulations can contain water, alcohol, polyethylene glycol ethers, and any other pharmaceutically acceptable solvents. Solubilizing agents and emulsifiers such as, without limitation: ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, fatty acid esters of sorbitan and mixtures thereof may also be present in said compound.

Additionally, oral compound of the proposed invention can include, without limitation, adjuvants such as wetting agents, emulsifying and suspending agents, sweetening, flavoring, and perfuming agents. When formulated as a suspension, said compound may contain the Cannabis sativa, Picralima nitida, Mitragyna speciose, Vinca major, and Psychotria species plants extracts and suspending agents, for example, without limitation: ethoxylated isostearyl alcohols, polyoxyethylene sorbitol, sorbitan esters, microcrystalline cellulose, aluminum metahydroxide, bentonite, agar-agar, tragacanth, and mixtures thereof

In one embodiment, the emulsifier may comprise a mixture of monoglyceride and diglyceride at a total concentration of 1% to 99% w/w and a carrageenan or mixture of carrageenans at a total concentration of 0.01% to 10% w/w. In another embodiment, the emulsifier may be present in a concentration range of 1% to 99%, 5% to 80%, 10% to 35%, 10% to 20%, or about 15%-25%% w/w.

Solid dosage forms suitable for oral administration include, capsules, tablets, pills, powders, and granules. In such solid dosage forms, the Cannabis sativa, Picralima nitida, Mitragyna speciose, Vinca major, and Psychotria species plants extracts can be used alone or in combination with one or more drugs that are mixed with at least one pharmaceutically acceptable excipient or carrier such as sodium citrate or dicalcium phosphate and/or fillers or extenders such as starches, lactose, sucrose, glucose, mannitol, and silicic acid; binders such as, for example, carboxymethylcellulose, alginates, gelatin, polyvinylpyrrolidone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents such as, for example, acetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof. For capsules, tablets and pills, the dosage form can also comprise buffering agents, such as acetic acid and Tris base.

Micellular or liposomal suspensions can be encapsulated with a variety of polymers, sugars, and chelating agents, to yield stable solid preparation. Encapsulation can take the form of cross linked polymers, trapping of the micells or liposomes within a non-crosslinked polymer network, or dispersed within the crystalline structure of sugar starches or protein molecules. These granules can be further processed to yield sublingual films, suppositories, dispersible powder, tablets, gel capsules, etc.

Solid dosages in the form of tablets, capsules, pills, and granules can be coated using compounds that accelerate or decrease the release of alkaloids. For instance, the proposed invention also encompasses solid dosage forms having enteric coatings, extended-release coatings, sustained-release coatings, delayed release coatings and immediate-release coatings. Methods used to coat solid dosage forms as well as the materials used to manufacture such coatings are well known in the pharmaceutical formulary art. The solid dosage forms can optionally contain opacity enhancing agents. According to one embodiment, the solid dosage form comprises an enteric coating that permits the release of Cannabis sativa, Picralima nitida, Mitragyna speciose, Vinca major, and Psychotria species plants extracts and/or their respective alkaloids, at a specific location within the gastrointestinal tract, optionally, in a delayed manner. Exemplary of such coating materials include glyceryl monostearate or glyceryl distearate may be employed, polymeric substances and waxes. The compound contemplated by this invention, alone or in combination with one or more drugs, can also be in micro-encapsulated foam, if appropriate, with one or more of the above-mentioned or other excipients.

In one embodiment, said compound is packaged into a gelatin capsule dosage form. In another embodiment, the compound is packaged into a non-gelatin capsule or an HPMC capsule. Said capsule can be a vegan based capsule or else. The compound disclosed herein includes a sustained release compound, an immediate release compound, or a combined sustained release fraction and immediate release fraction. In one embodiment, the therapeutic effect of the compound has a duration up to 4 hours, up to 6 hours, up to 8 hours, up to 10 hours, up to 12 hours, up to 14 hours, up to 16 hours, up to 18 hours, or up to 24 hours. In one embodiment, the compound disclosed herein comprises an immediate release fraction and a sustained release fraction, wherein the immediate release fraction contains a therapeutically effective amount of Cannabis sativa, Picralima nitida, Mitragyna speciose, Vinca major, and Psychotria species plants alkaloids and an edible oil; and wherein the sustained release fraction contains a therapeutically effective amount of Cannabis sativa, Picralima nitida, Mitragyna speciose, Vinca major, and Psychotria species plants alkaloids, and a mixture of emulsifiers and other pharmacologically inactive substances.

In another embodiment, a dietary compound, according to the present invention, is any ingestible preparation that contains the Cannabis sativa, Picralima nitida, Mitragyna speciose, Vinca major, and Psychotria species plants extracts as contemplated by this invention, where the pharmacologically inactive substance is a food product. The food product can be dried, cooked, boiled, lyophilized, baked, frozen, chilled, liquid, semi-liquid or prepared by any preparation used in food processing. Such food product can be, but not limited to: breads, teas, soups, cereals, salads, sandwiches, sprouts, vegetables, animal feed, pills and tablets, soft drinks, instant drinks, and any other human or animal food.

In yet another embodiment, a compound for parenteral injection comprises pharmaceutically acceptable sterile aqueous or non-aqueous solutions, dispersions, suspensions or emulsions as well as sterile powders for reconstitution into sterile injectable solutions or dispersions prior to use. Examples of suitable aqueous and non-aqueous carriers, diluents, solvents or vehicles include, without limitation, water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate.

In one embodiment, proper fluidity can be maintained, for example, by the use of coating materials such as lecithin, by the maintenance of the required particle size in the case of dispersions, and by the use of surfactants. The compound of the present invention can also contain adjuvants such as, but not limited to, preservatives, wetting agents, emulsifying agents, and dispersing agents. The compound for parenteral delivery generally includes isotonic agents such as sugars, sodium chloride, and the like. Prolonged absorption of the injectable pharmaceutical formulation can be brought about by the inclusion of agents which delay absorption such as aluminum monostearate and gelatin.

Injectable depot forms are made, in one embodiment, by forming microencapsule matrices of the drug in biodegradable polymers such as polylactide-polyglycolide. Depending upon the ratio of drug to polymer and the nature of the specific polymer employed, the rate of drug release can be controlled. Examples of other biodegradable polymers include poly-orthoesters and poly-anhydrides. Depot injectable formulations are also prepared by entrapping the drug in liposomes or microemulsions which are compatible with body tissues. The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Dosage forms for topical administration include, but are not limited to, ointments, creams, emulsions, lotions, gels, sunscreens and agents that favor penetration within the epidermis. Various additives, known to those skilled in the art, may be included in the topical formulations of the invention. Examples of additives include, but are not limited to, solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers. Examples of moisturizers include jojoba oil and evening primrose oil.

Suitable skin permeation enhancers are well known in the art and include lower alkanols, such as methanol ethanol and 2-propanol; alkyl methyl sulfoxides such as dimethylsulfoxide (DMSO), decylmethylsulfoxide (C10 MSO) and tetradecylmethyl sulfoxide; pyrrolidones, urea; N,N-diethyl-m-toluamide; C2-C6 alkanediols; dimethyl formamide (DMF), N,N-dimethylacetamide (DMA) and tetrahydrofurfuryl alcohol. Examples of solubilizers include, but are not limited to, hydrophilic ethers such as diethylene glycol monoethyl ether (ethoxydiglycol, available commercially as Transcutol) and diethylene glycol monoethyl ether oleate (available commercially as Softcutol); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol); alkyl methyl sulfoxides, such as DMSO; pyrrolidones, DMA, and mixtures thereof.

Prevention and/or treatment of a broader spectrum of infections can be achieved by inclusion of other antibiotics or anti-inflammatory agents or other active ingrediants, as well as other antibacterial and antifungal agents, for example, paraben, chlorobutanol, phenol sorbic acid, and the like, in the compounds of the invention.

One of ordinary skill will appreciate that effective amounts of the agents in the compound used in the methods of the invention can be determined empirically. It will be understood that, when administered to a patient, the total daily usage of the compound of the present invention will be decided by the attending physician within the scope of sound medical judgment. The specific therapeutically effective dose level for any patient will depend upon a variety of factors: the type and degree of the response to be achieved; the activity of the specific compound employed; the age, body weight, general health, sex and diet of the patient; the duration of the treatment; drugs used in combination or coincidental with the method of the invention; and like factors well known in the medical arts. An overview of the plants, their constituents, safety and efficacy are provided below.

There are certain side effects that were reported in connection with Picralima nitida and some other active ingredients employed in the proposed formulations. According to Erharuyi et al., (2014) the acute toxicological profile of the methanol fruit rind of Picralima nitida in rats revealed signs of toxic effect on the liver, kidneys and the lungs after prolonged exposure at high doses (1.5-6 g/kg) with LD50 values of 14.5 and 12.5 g/kg for male and female rats respectively. These effects were characterized by marked elevation in serum aspartate amino-transferase (AST), alanine amino-transferase (ALT), glucose, creatinine, total cholesterol and protein (Mabeku, Kouam, Paul, & Etoa, 2008).

Acute intraperitoneal toxicity tests of the basic alkaloidal fraction of Picralima nitida stem bark showed a dose-dependent toxicity characterized by inflammation and necrosis of the hepatocytes accompanied by reduction in neutrophilic count and a corresponding increase in lymphocytic count. Conjunctiva application showed no sign of reddening or irritation and dermal tests also showed no sensitization, inflammation or death in the animal models used (Fakeye, et al., 2004).

The acute and sub-chronic toxicity studies of the hydroethanol extract of Picralima nitida leaves (1-5 g/kg p.o. in mice) was carried out by Ilodigwe et al, (2012) the result of the study showed that Picralima nitida leaf extract caused no physical sign of toxicity within 24 hours after prolonged administration, assessment of hematological parameters showed that there was no significant elevation of hemoglobin concentration, packed cell volume and red blood cell count at lower doses. While at higher doses, there was a significant (P<0.05) elevation of white blood cell count and biochemical studies revealed a dose-and time-dependent elevation of serum AST, ALT, and serum alkaline phosphatase and a concomitant degeneration and rupture of the hepatocytes (Ilodigwe, Okoye, Mbagwu, Agbata, & Ajaghaku, 2012). Acute toxicity of the methanol pulp, seed and fruit rind extract of Picralima nitida on rats revealed a mild toxic effect with LD50 values of 7,071.0 mg/kg, 6,948.68 mg/kg and 1,364.91 mg/kg, respectively (Okonta & Aguwa, 2007).

Although Kratom seems to be safe when administered at 1-10 mg/kg doses (which represents a sub-chronic dose), according to Pantano (2016), after prolonged exposure to a 100 mg/kg dose, as demonstrated in the experiments on Sprague-Dawley rats conducted by Sabetghadam et al. (2013), it causes biochemical and hematological changes with histopathological alterations in several tissues (liver, kidney and brain). Another study reported that Kratom users consume about 67.5-75 mg of Kratom per day and that no adverse effects were shown while only after prolonged exposure to a higher dose of Kratom, clinical signs of toxicity were highlighted (Vicknasingam B. , Narayanan, Beng , & Mansor, 2010). Only a few papers (Kapp, Maurer, Auwarter, Winkelmann, & Hermann-Clausen, 2011), according to Pantano (2016), report liver damages or hepatotoxic sequelae related to Kratom use, and also in these cases, the authors highlighted the difficulties of a correlation between Kratom consumption and hepatic injuries, which was more likely to be associated with the extraction process of the alkaloids or to the presence of contaminants in the herbal products (Raffa, 2014). Moreover, causality has not yet been accurately established for Kratom, as CIOMS scale (RUCAM) has not been applied to suspected cases (Pantano, et al., 2016).

The pharmacological effects of Kratom are mainly attributed to its principal alkaloid mitragynine and 7-hydroxymitragynine. Mitragynine is the most abundant alkaloid in the leaves. Being a potent atypical opioid, it does have some liability of addiction. A study (Trakulsrichai, et al., 2013) was conducted to identify the characteristics of Kratom poisoning and withdrawal cases from Kratom exposure cases in Ramathibodi Poison Center (RPC), Thailand, during a five-year period. The study provides a retrospective review of Kratom exposure cases from the RPC toxic surveillance system. A total of 52 Kratom exposure cases were identified. There were Kratom poisoning cases (76.9%) and withdrawal cases (23.1%). Common presenting symptoms in the poisoning group were palpitation (22.5%), followed by seizure (17.5%). For the withdrawal group, the common presenting symptoms were myalgia (33.3%), insomnia (16.67%), fatigue (16.67%), and chest discomfort (16.67%). Trakulsrichai, et al., (2013) notes that there was a case of baby with withdrawal symptoms who was delivered from a chronic Kratom-abusing mother, suggesting possible exposure via the transplacental route. There were no deaths in either group. Kratom abuse can cause either poisoning or withdrawal. Most cases in both groups had good prognostic outcome (Trakulsrichai, et al., 2013).

According to Ramanathan, et al. (2015), several pharmacological studies were undertaken on rodents specifically for mitragynine. However, the mitragynine dose employed in these studies varied largely across rodent species, showing the following characteristics: analgesic (30-200 mg/kg), pharmacokinetics (20-50 mg/kg), toxicity (200-477 mg/kg). Others reported no toxicity even at mitragynine dose levels of 800-900 mg/kg in rodents. However, a study by Janchawee et al. (2007) demonstrated lethal effects after an oral administration of 200 mg/kg mitragynine in rats. The similar fatal effect was also observed after administration of 200 mg/kg alkaloid extract of Kratom to rats (Azizi, Ismail, Mordi, & Ramanathan, 2010).

It was also reported in a different study that a man who tried to abstain from Kratom had difficulty sleeping, wriggling sensation in the shoulders and the back, dragging sensation in the hips, bitemporal headache, became extremely weak and also had difficulty walking (Thuan, 1957). A study by Vicknasingam and colleagues (2010) also revealed that kratom produced mild side effects such as loss of weight, dehydration, constipation but no other medical problems were reported. However, prolonged use of kratom was reported to cause adverse effects which include nausea, diarrhea, vomiting, hallucinations, psychosis, agitation, dizziness, itching, sweating, dry mouth, respiratory depression, constipation, anorexia, increased urination, palpitations and weight loss (Suwanlert, 1975) (Jansen & Prast, 1988) (Babu, McCurdy, & Boyer, 2008) (Adkins, Boyer, & McCurdy, 2011). Other than adverse effects, there are no reports of mortalities following mitragynine or Kratom consumption alone, even after chronic and high dosage consumption (Ramanathan & Mansor, 2014).

In a series of 9 lethal cases from Sweden, both mitragynine (0.02-0.18 g/g) and O-desmethyltramadol (0.4-4.3 g/g) were detected in the post mortem blood samples of Krypton (powdered Kratom mixed with the u-opioid receptor agonist, O-desmethyltramadol, an active metabolite of Tramadol) users over a 1-year time. It was suggested that the addition of both, u-opioid receptor agonists, mitragynine and O-desmethyltramadol, to the herbal mixture may have caused the unintentional death. However, since no data for lethal doses in humans are available yet, the contribution of mitragynine to polytoxic causes of death is currently hard to estimate (Holler, et al., 2011).

In another embodiment of the proposed invention, the following preparation is made to treatment chronic pain: Dry solid ethanolic extract of Picralima nitida was pulverized and dispersed in diethyl ether in order to separate the polar from non-polar constituents. This non-polar solvent was meant to dissolve out the lipophilic constituents while leaving behind the hydrophilic ones (diethyl ether insoluble). Upon filtration the insoluble hydrophilic constituents were separated from the lipophilic constituents. The diethyl ether was evaporated off to recover the extract. The extracts were stored in the desiccator for one week.

The chitosan microspheres were prepared as follows: A quantity of chitosan powder was dissolved in a vessel containing acetic acid and Tween 80. Distilled water was added, and the solution was stirred vigorously. Sodium sulphate (Na₂SO₄) was then added to the chitosan solution with continuous stirring. Sonication for 15 min and 30 min centrifugation at 5,000 rpm respectively were subsequently carried out and the supernatant discarded. The sediment was re-suspended in distilled water 3 times to get rid of left-over acetic acid. The supernatant was decanted, and the sediment stored. Ethanolic extract of Picralima nitida was used instead of diethyl ether extract because it contained all the aqueous constituents. Picralima nitida powder was dispersed in equal weight of water prior to mixing with chitosan microspheres at a weight ratio of 1:6 and then sonicated for 30 minutes for complete interaction with the microspheres.

Granules were produced by wet granulation method using Carbosil as a bulking agent. Some quantity of Picralima nitida microspheres was mixed with some Carbosil and blended together. The mixture was thoroughly kneaded with mortar and pestle, screened through sieve 1.7 mm and the wet granules dried in a hot air oven at 50° C. for 1 hour. The dry granulation was obtained by screening the granules through 1.0 mm sieve. Hard gelatin capsules (no. 2) were automatically filled with 500 mg of granulation and stored in an amber bottle. Said process generally shows good granulation flow characteristics. The in-vitro drug release of Picralima nitida from the granulated microspheres showed a consistent pattern. The T50 and T85 values were 42 and 96 min respectively. The presence of Carbosil and microspheric entrapment were responsible for the slight slow release. Said preparation was used, in one embodiment, to treat moderate pain. One 500 mg pill was given to patients every 6 hours to manage pain.

In another embodiment of the proposed invention, the following topical preparation is made to treat fungal nail infection. Cream formulation with 10% concentration of Picralima nitida extract was prepared using Aqueous Cream BPC as base material and 10% Propylene Glycol (PG) as humectant. This formulation appeared to generally have the highest effectiveness against the microorganisms. Said cream had also exhibited high effectiveness against bacterial infections of skin when applied 4 times a day, with most of the signs/symptoms disappearing after 2-3 weeks of medication.

The potential commercial uses of the disclosed preparations include, for example, protective/prophylactic and medical uses. The compounds of the invention can also be administered by a variety of other routes, including mucosal, subcutaneous and intramuscular administration, and may comprise a variety of carriers or excipients known in the formulary art, such as, non-toxic solid, semisolid or liquid filler, diluent, encapsulating material and formulation auxiliaries that are pharmaceutically acceptable.

The features disclosed in the foregoing description, or the following claims, or the accompanying drawings, expressed in their specific forms or in terms of a means for performing the disclosed function, or a method or system for attaining the disclosed result, as appropriate, may separately, or in any combination of such features, be utilized for realizing the invention in diverse forms thereof.

While various embodiments of the present invention have been described above, it should be understood that they have been presented by way of example only, and not limitation. It will be understood by those skilled in the art that various changes in form and details may be made therein without departing from the spirit and scope of the invention as defined in the appended claims. Thus, the breadth and scope of the present invention should not be limited by any of the above-described exemplary embodiments but should be defined in accordance with the following claims and their equivalents.

### REFERENCES

Adkins, J. E., Boyer, E. W., & McCurdy, C. R. (2011). Mitragyna speciosa, a psychoactive tree from Southeast Asia with opioid activity. Current Topics in Medicinal Chemistry, 11, 1165-1175.
Amador, T. A., Elisabetsky, E., & Onofre de Souza, D. (1996). Effects of Psychotria colorata alkaloids in brain opioid system. Neurochemical Research, 21(1), pp. 97-102. doi:10.1007/BF02527677
Amador, T. A., Verotta, L., Nunes, D. S., & Elisabetsky, E. (2001, January). Antinociceptive Profile of Hodgkinsine. Planta Medica, 66(8), pp. 770-2. doi:10.1055/s-2000-9604
Amador, T. A., Verotta, L., Nunes, D. S., & Elisabetsky, E. (2001, May). Involvement of NMDA receptors in the analgesic properties of psychotridine. Phytomedicine, 8(3), pp. 202-6. doi:10.1078/0944-7113-00025
Ameh, S. J., Tarfa, F., Abdulkareem, T. M., Ibe, M. C., Onanuga, C., & Obodozie, O. O. (2010, April). Physicochemical Analysis of the Aqueous Extracts of Six Nigerian Medicinal Plants. Tropical Journal of Pharmaceutical Research, 9(20), pp. 119-125. doi:10.4314/tjpr.v9i2.53698
Azizi, J., Ismail, S., Mordi, M. N., & Ramanathan, S. (2010). In vitro and in vivo effects of three different Mitragyna speciosa Korth leaf extracts on phase II drug metabolizing enzymes—glutathione transferases (GSTs). Molecules, 15, 432-441.
Babu, K. M., McCurdy, C. R., & Boyer, E. W. (2008). Opioid receptors and legal highs: Salvia divinorum and Kratom. Clinical Toxicology, 46(2), 146-152.
Benyamin, R., Trescot, A. M., Datta, S., Buenaventura, R., Adlaka, R., Sehgal, N., . . . Vallejo, R. (Mar. 11, 2008). Opioid complications and side effects. Pain Physician., S105-20.
Blake, A., Wan, B. A., Malek, L., DeAngelis, C., Diaz, P., Lao, N., . . . O'Hearn, S. A. (Dec. 6, 2017). Selective review of medical cannabis in cancer pain management. Ann Palliat Med., S215-S222. doi:10.21037/apm.2017.08.05.
Both, F. L., Kerber, V. A., Henriques, A. T., & Elisabetsky, E. (2002). Analgesic Properties of Umbellatine from Psychotria umbellata. Pharmaceutical Biology, 40(5), pp. 336-341. doi:10.1076/phbi.40.5.336.8453
Brenneisen, R. (2007). Chemistry and Analysis of Phytocannabinoids and Other Cannabis Constituents. In R. Brenneisen, Chemistry of Cannabis Constituents (pp. 17-49). Totowa: Humana Press. doi:https://doi.org/10.1007/978-1-59259-947-9_2
Chittrakarn, S., Keawpradub, N., Sawangjaroen, K., Kansenalak, S., & Benjamas, J. (2010). The neuromuscular blockade produced by pure alkaloid, mitragynine and methanol extract of kratom leaves (Mitragyna speciosa Korth.). Journal of Ethnopharmacology, 129, 344-349. Retrieved from http://entheology.com/wp-content/uploads/kratom-research/2010_std008.pdf
Dapaah, G., Koffuor, G. A., Mante, P. K., & Ben, I. O. (2016, Mar-Apr). Antitussive, expectorant and analgesic effects of the ethanol seed extract of Picralima nitida (Stapf) Th. & H. Durand. Res Pharm Sci., 11(2), pp. 100-112. Retrieved from https://www.ncbi.nlm.nih.gov/pmc/articles/PMC4852654/?report=printable
Duflos, A., Fahy, J., Thillaye du Boullay, V., Barret, J.-M., & Hill , B. (1997). U.S. Pat. No. 6,127,377.
Duwiejua, M., Woode, E., & Obiri, D. D. (2002, Jun). Pseudo-akuammigine, an alkaloid from Picralima nitida seeds, has anti-inflammatory and analgesic actions in rats. J Ethnopharmacol., 81(1), pp. 73-9. Retrieved from https://www.ncbi.nlm.nih.gov/pub med/12020930
Elisabetsky, E., Amador, T. A., Albuquerque, R. R., Nunes, D. S., & Carvalho, A. T. (1995, Oct). Analgesic activity of Psychotria colorata (Willd. ex R. & S.) Muell. Arg. alkaloids. Journal of Ethnopharmacology, 48(2), pp. 77-83. doi:10.1016/0378-8741(95)01287-N
Erharuyi, O., & Falodun, A. (2012, Sept). Free Radical Scavenging Activities of Methanol

Extract and Fractions of Picralima nitida (Apoceanacea). J. Appl. Sci. Environ. Manage., 16(3), pp. 291-294. Retrieved from https://www.ajol.info/index.php/jasem/article/viewFile/90947/80366
Erharuyi, O., Falodun, A., & Langer, P. (2014). Medicinal uses, phytochemistry and pharmacology of Picralima nitida (Apocynaceae) in tropical diseases: A review. Asian Pacific Journal of Tropical Medicine, pp. 1-8.
Ezeamuzie, I. C., Ojinnaka, M. C., Uzogara, E. O., & Oji, S. E. (1994, Mar). Anti-inflammatory, antipyretic and anti-malarial activities of a West African medicinal plant—Picralima nitida. Afr J Med Med Sci., 23(1), pp. 85-90. Retrieved from https://www.ncbi.nlm.nih.gov/pub med/7839951
Fakeye, T. O., Awe, S. O., Odelola, H. A., Ola-Davies, O. E., Itiola, O. A., & Obajuluwa, T. (2004). Evaluation of valuation of toxicity profile of an alkaloidal fraction of the stem bark of Picralima nitida (fam. Apocynacaes). J Herb Pharmacother., 4(3), pp. 37-45.doi:10.1080/J157v04n0304
Fakeye, T. O., Itiola, O. A., & Odelola, H. A. (2000, Aug). Evaluation of the antimicrobial property of the stem bark of Picralima nitida (Apocynaceae). Phytother Res., 14(5), pp. 368-70. Retrieved from https://www. ncbi.nlm.nih.gov/pubmed/10925406
Falcon, E., Browne, C. A., Leon, R. M., Fleites, V. C., Sweeney, R., Kirby, L. G., & Lucki, I. (2016). Antidepressant-like Effects of Buprenorphine are Mediated by Kappa Opioid Receptors. Neuropsychopharmacology, 41(9), 2344-51. doi:10.1038/npp.2016.38
Formukong, E. A., Evans, A. T., & Evans, F. J. (1988, August). Analgesic and antiinflammatory activity of constituents of Cannabis sativa L. PubMed, 12(4), 361-371. Retrieved from https://www.ncbi.nlm.nih.gov/pub med/3169967
Forssen, E., Cox, G., & Hair, D. (1994). U.S. Pat. No. 5,714,163.
Geiser, F., Keenan, J., Rossi, R., Sanchez, A., & Whelan, J. (2004). U.S. Pat. No. 7,449,589.
Guy, L., & LeMenez, P. (1992, Mar). Akuammine and dihydroakuammine, two indolomonoterpene alkaloids displaying affinity for opioid receptors. J Nat Prod., 55(3), pp. 380-4. Retrieved from https://www.ncbi.nlm.nih.gov/pub med/1317407
Haney, M., Gunderson, E. W., Rabkin, J., Hart, C. L., Vosburg, S. K., Comer, S. D., & Foltin, R. W. (2007, Aug 15). Dronabinol and marijuana in HIV-positive marijuana smokers. Caloric intake, mood, and sleep. J Acquir Immune Defic Syndr., 45(5), 545-54.
Harizal, S. N., Mansor, S. M., Hasnan, J., Tharakan, J. K., & Abdullah, J. (2010). Acute toxicity study of the standardized methanolic extract of Mitragyna speciosa Korth in rodent. Journal of Ethnopharmacology, 2, 404-409.
Hassana, Z., Muzaimi, M., Navaratnama, V., Yusoff N. H., Suhaimi, F. W., Vadivelua, R., Müller, C. P. (2013). From Kratom to mitragynine and its derivatives: Physiological and behavioural effects related to use, abuse, and addiction. Neuroscience and Biobehavioral Reviews, 37, 138-151.
Holdcroft, A., Smith, M., & Jacklin, A. (1997). Pain relief with oral cannabinoids in familial Mediterranean fever. Anaesthesia, 12, 44-49.
Holler, J. M., Vorce, S. P., McDonough-Bender, P. C., Magluilo Jr, J., Solomon, C. J., & Levine, B. (2011). A drug toxicity death involving propylhexedrine and mitragynine. Journal of Analytical Toxicology, 35, 54-59.
Igwe, O. U., & Mgbemena, M.-A. N. (2014). Chemical Profiling and Antibacterial Activity

Screening of The Leaves of Picralima Nitida (Apocynaceae). International Journal of Medicinal Chemistry & Analysis, 4(3), pp. 155-161.
Ilodigwe, E. E., Okoye, G. O., Mbagwu, I. S., Agbata, C. A., & Ajaghaku, D. L. (2012). Safety Evaluation of Ethanol Leaf Extract of Picralima nitida Stapf (Apocynaceae). International Journal of Pharmacology and Therapeutics, 2(4), pp. 6-17.
Institute of Medicine. (1999). Cannabinoids and animal physiology. In I. o. Medicine, Marijuana and Medicine: Assessing the Science Base (pp. 2.1-2.47). Washington, D.C.: National Academy Press.
Jain, A. K., Ryan, J. R., & McMahon, F. G. (1981). Evaluation of intramuscular levonantradol and placebo in acute postoperative pain. Journal of Clinical Pharmacology, 21((suppl. 8-9)), 320S-326S.
Janchawee, B., Keawpradub, N., Cittrakarn, S., Prasettho, S., Wararatananurak, P., & Sawangjareon, K. (2007). A high-performance liquid chromatographic method for determination of mitragynine in serum and its application to a pharmacokinetic study in rats. Biomed. Chromatogr., 21, 176-183.
Janet, M. M., Le Men, J., Aghoramurthy, K., & Robinson, R. (1955). The Identity of Vincamajoridine and Akuammine. Oxford: Laboratory o/Galenic Pharmacy, Faculty of Pharmacy, University of Paris, and Dyson Perrins Laboratory, Oxford University. Retrieved from https://link.springer.com/content/pdf/10.1007%2FBF02159911.pdf
Jannic, V., Guéritte, F., Laprévote, O., Serani, L., Martin, M. T., Sévenet, T., & Potier, P. (1999, Jun). Pyrrolidinoindoline alkaloids from Psychotria oleoides and Psychotria lyciiflora. J Nat Prod, 62(6), pp. 838-43. doi:10.1021/np9805387
Jansen, K. L., & Prast, C. (1988). Ethnopharmacology of kratom and the Mitragyna alkaloids. Journal of Ethnopharmacology, 23, 115-119.
Kalant, H. (2004). Adverse effects of cannabis on health: an update of the literature since 1996. Prog Neuropsychopharmacol Biol Psychiatry, 28(5), 849-863. doi:10.1016/j.pnpbp.2004.05.027
Kapp, F. G., Maurer, H. H., Auwarter, V., Winkelmann, M., & Hermann-Clausen, M. (2011). Intrahepatic cholestasis following abuse of powdered kratom (Mitragyna speciosa). J. Med. Toxicol., 7, 227-231. doi:10.1007/s13181-011-0155-5
Karniol, I. G., Shirakawa, I., Kasinski, N., Pfeferman, A., & Carlini, E. A. (1974, Sep). Cannabidiol interferes with the effects of Δ9-tetrahydrocannabinol in man. European Journal of Pharmacology, 28(1), pp. 172-177. doi:10.1016/0014-2999(74)90129-0
Kluger, B., Triolo, P., Jones, W., & Jankovic, J. (2015). The Therapeutic Potential of Cannabinoids for Movement Disorders. PubMed Central. doi:10.1002/mds.26142
Koff, N. G., Emma, A. A., & Stephan, D. K. (2014). Evaluation of Picralima nitida acute toxicity in the mouse. Int J Res Pharm Sci., 4(3), pp. 18-22. Retrieved from https://www.ijrpsonline.com/down_2036.php
Kruegel, A. C., Gassaway, M. M., Kapoor, A., Váradi, A., Majumdar, S., Filizola, M., . . . Sames, D. (2016). Synthetic and Receptor Signaling Explorations of the Mitragyna. J Am Chem Soc., 138(21), 6754-64. doi:10.1021/jacs.6b00360
Kumarnsit, E., Keawpradub, N., & Nuankaew, W. (2006). Acute and long-term effects of alkaloid extract of Mitragyna speciosa on food and water intake and body weight in rats. Fitoterapia, 77, 339-345.
Langford, R. M., Mares, J Novotna, A., Vachova, M., Novakova, I., Notcutt, W., & Ratcliffe, S. A. (2013). A double-blind, randomized, placebo-controlled, parallel-group study of THC/CBD oromucosal spray in combination with the existing treatment regimen, in the relief of central neuropathic pain in patients with multiple sclerosis. J Neurol., 260(4), 984-97. doi:10.1007/s00415-012-6739-4
Lewin, G., Le Ménez, P., Rolland, Y., Renouard, A., & Giesen-Crouse, E. (1992, March). Akuammine and dihydroakuammine, two indolomonoterpene alkaloids displaying affinity for opioid receptors. J. Nat. Prod., 55(3), pp. 380-384. doi:10.1021/np50081a017
Lynch, M. E., Cesar-Rittenberg, P., & Hohmann, A. G. (2014). A double-blind, placebo-controlled, crossover pilot trial with extension using an oral mucosal cannabinoid extract for treatment of chemotherapy-induced neuropathic pain. J Pain Symptom Manage., 47(1), 166-73. doi:10.1016/j.jpainsymman.2013.02.018
Mabeku, L. K., Penlap, B. V., Kouam, J Ngadjui, B. T., Fomum, Z. T., & Etoa, F. X. (2006,Oct). Evaluation of antidiarrhoeal activity of the fruit-rind of Picralima nitida (Apocynaceae). African Journal of Traditional, Complementary and Alternative Medicines, 3(4), pp. 66-73. doi:10.4314/ajtcam.v3i4.31178
Mabeku, L., Kouam, J., Paul, A., & Etoa, F. X. (2008). Phytochemical screening and toxicological profile of methanolic extract of Picralima nitida fruit rind (Apocynaceae). Toxicol Environ chem, 90(4), pp. 815-828. Retrieved from httpsliwww.tandfonline.com/doi/abs/10.1080/02772240701747556
Matsumoto, K., Hatori, Y., Murayama, T., Tashima, K., Wongseripipatana, S., Misawa, K., . . . Horie, S. (2006). Involvement of u-opioid receptors in antinociception and inhibition of gastrointestinaltransitinduced by 7-hydroxymitragynine, isolated from Thai herbal medicine Mitragyna speciosa. Journal of Pharmacology, 549, 63-70.
Maurer, M., Henn, V., & Dittrich, A. (1990). Delta-9-tetrahydrocannabinol shows antispastic and analgesic effects in a single case double-blind trial. European Archives of Psychiatry and Clinical Neuroscience, 240, 1-4.
Maurice, M., Iwu, D. L., Klayman, J. E., Jackson, J. E., Tally, J. D., & Andersen, S. L. (1991). U.S. Pat. No. 5,290,553.
Menzies, J. R., Paterson, S. J Duwiejua, M., & Corbett, A. D. (1998). Opioid activity of alkaloids extracted from Picralima nitida (fam. Apocynaceae). European Journal of Pharmacology, 350, pp. 101-108.
No authors listed. (2011). Prescrire International. Adverse effects of cannabis, 20(112), 1823. Retrieved from https://www.ncbi.nlm.nih.gov/pub med/21462790
Noyes, R. J., Brunk, S. F., & Avery, D. A. (1975). The analgesic properties of delta-9-tetrahydrocannabinol and codeine. Clinical Pharmacology and Therapeutics, 18, 84-89.
Noyes, R. J., Brunk, S., & Baram, D. A. (1975). Analgesic effect of delta-9-tetrahydrocannabinol. Journal of Clinical Pharmacology, 15, 139-143.
Okada, Y., Tsuda, Y., Salvadori, S., & Lazarus, L. H. (2012). Developmental Potential for

Endomorphin Opioidmimetic Drugs. International Journal of Medicinal Chemistry, 2012, p. 10 pages. doi:10.1155/2012/715123
Okokon, J. E., Antia, B. S., Igboasoiyi, A. C., Essien, E. E., & Mbagwu, H. O. (May 22, 2007). Evaluation of antiplasmodial activity of ethanolic seed extract of Picralima nitida. J Ethnopharmacol, 111(3), pp. 464-7. doi:10.1016/j.jep.2006.12.016
Okonta, J. M., & Aguwa, C. N. (2007). Evaluation of Hypoglycemic Activity of Glycosides and Alkaloids Extracts of Picralima nitida Stapf (Apocynaceae) Seed. International Journal of Pharmacology, 3(6), pp. 505-509. doi:ijp.2007.505.509
Okonta, M. J., Adibe, O. M., & Ubaka, M. C. (2011, January). Antiulcer activity of methanolic extract and fractions of Picralima nitida seeds(Apocynacaea) in rats. Asian Pacific Journal of Tropical Medicine, 4(1), pp. 13-15. doi:10.1016/S1995-7645(11)60023-0
Pantano, F., Tittarelli, R., Mannocchi, G., Zaami, S., Ricci, S., Giorgetti, R., . . . Marinelli, E. (2016). Hepatotoxicity Induced by “the 3Ks”: Kava, Kratom and Khat. Int J Mol Sci., 17(4), 580. doi:10.3390/ijms17040580
Pertwee, R. G. (2008, January). The diverse CB1 and CB2 receptor pharmacology of three plant cannabinoids: Δ9-tetrahydrocannabinol, cannabidiol and Δ9-tetrahydrocannabivarin. British Pharmacological Society, 153(2), pp. 199-215. doi:10.1038/sj.bjp.0707617
Porto, D. D., Henriques, A. T., & Fett-Neto, A. G. (2009). Bioactive Alkaloids from South American Psychotria and Related Species. The Open Bioactive Compounds Journal, 2, pp. 29-36. doi:10.2174/1874847300902010029
Purintrapiban, J., Keawpradub, N., Kansenalak, S., Chittrakarn, S., Janchawee, B., & Sawangjaroen, K. (2011). Study on glucose transport in muscle cells by extracts from Mitragyna speciosa (Korth) and mitragynine. Natural Product Research, 25(15), 1379-1387.
Raffa, R. B. (2014). Kratom and Other Mitragynines. In R. B. Raffa, The Chemistry and Pharmacology of Opioids from a Non-Opium Source. Boca Raton: CRC Press Taylor & Francis Group.
Ramanathan, S., & Mansor, S. M. (2014). Toxicology of Mitragynine and analogues. In S. Ramanathan, S. M. Mansor, & R. B. Raffa (Ed.), The Chemistry and Pharmacology of Opioids from a Non-Opium (1st ed., pp. 281-292). CRC Press.
Ramanathan, S., Parthasarathy, S., Murugaiyah, V., Magosso, E., Tan, S. C., & Mansor, S. M. (2015). Understanding the Physicochemical Properties of Mitragynine, a Principal Alkaloid of Mitragyna speciosa, for Preclinical Evaluation. Molecules, 20, 4915-4927. doi:10.3390/molecules20034915
Robson, P. (2001). Therapeutic aspects of cannabis and cannabinoids. The British Journal of Psychiatry, 178, 107-115. Retrieved from http://www.ukcia.org/research/Thereputic/Therapeut.htm#35
Russo, E., & Guy, G. W. (Oct. 4, 2006). A tale of two cannabinoids: the therapeutic rationale for combining tetrahydrocannabinol and cannabidiol. Med Hypotheses, 66(2), pp. 234-46. Retrieved from https://www.ncbi.nlm.nih.gov/pub med/16209908
Sabetghadam , A., Ramanathan , S., Sasidharan, S., & Mansor, S. M. (2013). Subchronic exposure to mitragynine, the principal alkaloid of Mitragyna speciosa, in rats. J. Ethnopharmacol, 146, 815-823. doi:10.1016/j.jep.2013.02.008
Saxton, J. E. (2014). Alkaloids of Picralima Nitida. In R. H. Manske, The Alkaloids: Chemistry and Physiology (pp. 119-157). Elsevier.
Shaik Mossadeq, W. M., Sulaiman, M. R., Tengku Mohamad, T. A., Chiong H. S., Baharuldin, M. T. H., Israf, D. A., M. T., Baharuldin, M. T., & Israf, D. A. (2009). Anti-inflammatory and antinociceptive effects of Mitragyna speciosa Korth methanolic extract. Medical Principles and Practice, 18, 378-384.
Shittu, H., Gray, A., Furman, B., & Young, L. (2010, March). Glucose uptake stimulatory effect of akuammicine from Picralima nitida (Apocynaceae). Phytochemistry Letters, 3(1), pp. 53-55. doi:10.1016/j.phyto1.2009.11.003
Singh, S., Jarial, R., & Kanwar, S. S. (2013). Therapeutic effect of herbal medicines on obesity: herbal pancreatic lipase inhibitors. Wudpecker J. Med. Plants, pp. 53-65.
Sukhdev, S., & Shamsher, K. S. (2016). Antilipase activity guided fractionation of Vinca major. Journal of King Saud University—Science. doi:10.1016/j.jksus.2017.03.005
Sukhdev, S., & Shamsher, K. S. (2017). Antilipase activity guided fractionation of Vinca major. Journal of King Saud University—Science. doi:10.1016/j.jksus.2017.03.005
Suwanlert, S. (1975). A study of kratom eaters in Thailand. Bulletin on Narcotics, 27(3), 21-27.
Tane, P., Tene, M., & Sterner, O. (2002). Picranitine, a new indole alkaloid from picralima nitida (APOCYNACEAE). Bulletin of the Chemical Society of Ethiopia, 16(2), pp. 165-169. Retrieved from https://www.ajol.info/index.php/bcse/article/view/20939
Teugwa, C. M., Mejiato, P. C., Zofou, D., Tchinda, B. T., & Boyom, F. F. (Jul. 15, 2013). Antioxidant and antidiabetic profiles of two African medicinal plants: Picralima nitida (Apocynaceae) and Sonchus oleraceus (Asteraceae). BMC Complement Ahern Med., 13, p. 175. doi:10.1186/1472-6882-13-175
Thame , N. (1986). U.S. Pat. No. 4,853,213.
Thuan, L. C. (1957). Addiction to Mitragyna speciosa. Proceedings of the Alumni Association, 10, 322-324.
Trakulsrichai, S., Tongpo, A., Sriapha, C., Wongvisawakorn , S., Rittilert, P., Kaojarern, S., & Wananukul, W. (2013). Kratom abuse in Ramathibodi Poison Center, Thailand: a five-year experience. J Psychoactive Drugs. 2013 Nov Dec; 45(5):, 45(5), 404-8. doi:10.1080/02791072.2013.844532
Tsuchiya, S., Miyashita, S., Yamamoto, M., Horie, S., Sakai, S. I., Aimi, N., . . . Watanabe, K. (2002). Effect of mitragynine, derived from Thai folk medicine on gastric acid secretion through opioid receptor in anesthetized rats. European of Pharmacology, 443, 185-188. Ubulom, M. E., Imandeh, N. G., Udobi, C. E., & IIya, I. (2012). Larvicidal and Antifungal

Properties of Picralima nitida (Apocynaceae) Leaf Extracts. European Journal of Medicinal Plants, 2(2), pp. 132-139. Retrieved from http://www.journalrepository.org/media/journals/EJMP 13/2012/Mar/1331463536-Ubulometal_2011EJMP869.pdf
Verotta, L., Orsini, F., Sbacchi, M., Scheildler, M. A., Amador, T. A., & Elisabetsky, E. (2002, July). Synthesis and antinociceptive activity of chimonanthines and pyrrolidinoindoline-Type alkaloids. Bioorganic & Medicinal Chemistry, 10(7), pp. 2133-2142. doi:10.1016/S0968-0896(02)00078-0
Vicknasingam, B., Narayanan, S., Beng , G. T., & Mansor, S. M. (2010). The informal use of ketum (Mitragyna speciosa) for opioid withdrawal in the northern states of peninsular Malaysia and implications for drug substitution therapy. Int. J. Drug Policy, 21, 283-288. doi:10.1016/j.drugpo.2009.12.003
Vicknasingam, B., Narayanan, S., Beng, G., & Mansor, S. (2010). The informal use of ketum (Mitragyna speciosa) for opioid withdrawal in the northern states of peninsular Malaysia and implications for drug substitution therapy. Int J Drug Policy, 21(4), 283-8. doi:10.1016/j.drugpo
Walker, L. A., Harland, E. C., Best, A. M., & ElSohly, M. A. (1999). Δ9-THC Hemisuccinate in Suppository Form as an Alternative to Oral and Smoked THC. In S. K. Nahas G. G., Marihuana and Medicine (pp. 123-135). Totowa: Humana Press. doi:10.1007/978-1-59259-710-9_13
Wren, R. C. (1988). Potter's New Cyclopaedia of Botanical Drugs and Preparations. The C.W. Daniel Company Ltd.
[symbol] , [symbol] , [symbol] , [symbol] . (2013). Korea Patent No. 101430354.
[symbol] , [symbol] , [symbol] . (2014). Korea Patent No. 20150096176.
[symbol] , & [symbol] . (2015). China Patent No. 104926840.
[symbol] , [symbol] [symbol] (2009). CN Patent No. 102,048,857. Retrieved from https://www.google.com/patents/CN102048857A

What claimed is:

1. A method for reducing inflammation and pain in subjects in need thereof, the method comprises of administering to the subject at least once in 24 hours at least 1.5 mg by dry mass per one kg of body weight in 24 hours, but not more than 16,000 mg by dry mass per one kg of body weight in 24 hours of a compound, comprising of an extract of Picralima nitida plant and at least one pharmacologically inactive substance; and where said extract contains at least one of not less than 0.02% of akuammine or not less than 0.02% of dihydroakuammine, or not less than 0.001% of vincamajoridine, or not less than 0.001% of akuammidine or not less than 0.001% of pseudoakuammigine, or not less than 0.001% of akuammicine.

2. A method for reducing symptoms associated with an upper respiratory tract infection (common cold), a seasonal allergy reaction, or an acute respiratory illness (flu) of viral or bacterial origin in subjects in need thereof, the method comprises of administering to the subject at least once in 24 hours at least 0.5 mg by dry mass per one kg of body weight in 24 hours, but not more than 16,000 mg by dry mass per one kg of body weight in 24 hours of a compound, comprising of at least 5% of an extract of Picralima nitida plant and at least one pharmacologically inactive substance.

3. A method for producing antitussive, expectorant, and bronchodilating effects in subjects in need thereof, the method comprises of administering to the subject at least once in 24 hours at least 1.5 mg by dry mass per one kg of body weight in 24 hours, but not more than 16,000 mg by dry mass per one kg of body weight in 24 hours of a compound, comprising of an extract of Picralima nitida plant and at least one pharmacologically inactive substance; and where said extract contains at least one of not less than 0.02% of akuammine or not less than 0.02% of dihydroakuammine, or not less than 0.001% of vincamajoridine, or not less than 0.001% of akuammidine, or not less than 0.001% of pseudoakuammigine, or not less than 0.001% of akuammicine or not less than 0.001% of psychotridine; and where said compound contains at least a trace amount of saponins.

4. A method for reducing dermatophytosis and dermatomycosis in subjects in need thereof the method comprises of topically administering to the subject at least once in 24 hours at least 1.5 mg of compound in 24 hours, containing at least 3% of crude extract of Picralima nitida plant, and where said compound contains at least one pharmacologically inactive substance and a humectant.

5. A method for reducing phytosis and mycosis of at least of the following types: superficial, cutaneous, subcutaneous systemic due to primary pathogens, and subcutaneous systemic due to opportunistic pathogens in subjects in need thereof, the method comprises of administering orally or by injection to the subject at least once in 24 hours at least 3 mg by dry mass per one kg of body weight in 24 hours, but not more than 16,000 mg by dry mass per one kg of body weight in 24 hours, a compound containing at least 5% of Picralima nitida plant extract, and where said compound contains at least one pharmacologically inactive substance.

6. A method for reducing obesity and body weight in subjects in need thereof the method comprises of administering to the subject at least once in 24 hours at least 1.5 mg by dry mass per one kg of body weight in 24 hours, but not more than 16,000 mg by dry mass per one kg of body weight in 24 hours of a compound comprising of an extract of Picralima nitida plant and at least one pharmacologically inactive substance; and where said extract contains at least 0.001% of akuammine or vincamajoridine.

7. A method for symptomatic treatment of diabetes in subjects in need thereof the method comprises of administering to the subject at least once in 24 hours at least 0.8 mg by dry mass per one kg of body weight in 24 hours, but not more than 16,000 mg by dry mass per one kg of body weight in 24 hours of a compound, comprising of at least one pharmacologically inactive substance, and at least 5% of a glycosides extract of Picralima nitida plant or an alkaloid extract of Picralima nitida plant.

8. A method for reducing gastritis, acid overproduction, acid reflux, and peptic ulcers in subjects in need thereof the method comprises of administering to the subject at least once in 24 hours at least 0.5 mg by dry mass per one kg of body weight in 24 hours, but not more than 16,000 mg by dry mass per one kg of body weight in 24 hours of a compound comprising of at least 3% extract of Picralima nitida plant and where said compound contains at least one pharmacologically inactive substance.

9. A compound for reducing symptoms associated with an upper respiratory tract infection (common cold), a seasonal allergy reaction, or an acute respiratory illness (flu) of viral or bacterial origin in subjects in need thereof, comprising of an extract of Picralima nitida plant and at least one pharmacologically inactive substance; and where said extract contains at least one of not less than 0.001% of akuammine, dihydroakuammine, akuammidine, pseudoakuammigine, akuammicine, vincamajoridine alkaloids; and where said compound contains at least a trace amount of one or more other plurality of alkaloids.

10. A compound for at least one of
reducing inflammation and pain;
reducing symptoms associated with an upper respiratory tract infection (common cold), a seasonal allergy reaction, or an acute respiratory illness (flu) of viral or bacterial origin;
producing antitussive, expectorant, and bronchodilating effects;
reducing dermatophytosis and dermatomycosis;
reducing phytosis and mycosis of at least of the following types: superficial, cutaneous, subcutaneous systemic due to primary pathogens, and
subcutaneous systemic due to opportunistic pathogens;
reduction of obesity and body weight;
producing hypoglycemic effect;
reducing gastritis, acid overproduction, acid reflux, and peptic ulcers in subjects in need thereof, comprising of an extract of Picralima nitida plant and at least one pharmacologically inactive substance; and where said compound contains at least 0.001% of total compound of at least one of Mitragyna speciose extract, Cannabis sativa extract, Psychotria species plant extract.

11. A compound for at least one of
reducing inflammation and pain;
reducing symptoms associated with an upper respiratory tract infection (common cold), a seasonal allergy reaction, or an acute respiratory illness (flu) of viral or bacterial origin;
producing antitussive, expectorant, and bronchodilating effects;
reducing dermatophytosis and dermatomycosis;
reducing phytosis and mycosis of at least of the following types: superficial, cutaneous, subcutaneous systemic due to primary pathogens, and
subcutaneous systemic due to opportunistic pathogens;
reduction of obesity and body weight;
producing hypoglycemic effect;
reducing gastritis, acid overproduction, acid reflux, and peptic ulcers in subjects in need thereof, comprising of at least one pharmacologically inactive substance; and at least 0.001% of total compound a natural or synthetic or semi-synthetic at least one of akuammine, dihydroakuammine, akuammidine, pseudoakuammine, akuammicine, akuammigine, pseudoakuammigine, akuammiline and akuammenine, picraphylline, picracine, picraline, picralicine, picratidine, picranitine, burnamine, pericalline and pericineby, reserpinine, majoridine, vincamajoridine, vincamine, vincamajine, perivincine, pubescine, vinine, vincawajine, majorinine, 10-methoxyvinorineor; and
where said compound contains at least 0.01% of total compound a natural or synthetic or semi-synthetic at least one of indole monoterpene-type alkaloid, pyrrolidinoindoline-type alkaloid, mitragynine, pseudoindoxyl, 7-hydroxymitragynine.

12. The compound of claim 9, where said compound is for at least one of reducing inflammation and pain; producing antitussive, expectorant, and bronchodilating effects; reducing dermatophytosis and dermatomycosis; reducing phytosis and mycosis of at least of the following types: superficial; cutaneous, subcutaneous systemic due to primary pathogens, and subcutaneous systemic due to opportunistic pathogens; reducing obesity and body weight; producing hypoglycemic effect; reducing gastritis, acid overproduction, acid reflux, and peptic ulcers in subjects in need thereof.

13. The method and compound of claims 1, 2, 3, 4, 5, 6, 7, 8, 9, 10 and 11, where said pharmacologically inactive substance is at least one of a medically acceptable carrier, a skin penetration enhancer, an absorption enhancer, a stabilizer, a solvent, pharmaceutically acceptable fixed oil, a lipid carrier, a polymer, a stabilizing agent, a disintegrant, a lubricant, a diluent, an adjuvant, an emulsifier, a preservative, a colorant, a flavor imparting agent, an acid, a Tris base, sodium carbonate, or a combination thereof.

14. The method and compound of claims 1, 2, 3, 4, 5, 6, 7, 8, 9, 10 and 11, where said compound is administered in a sustained release, or extended release, or a combined sustained release and extended release dosage forms, or in an immediate release dosage forms, or a combined sustained release and immediate release dosage forms, or a combination thereof.

15. The method and compound of claims 1, 2, 3, 4, 5, 6, 7, 8, 9, 10 and 11, where said compound is administered by a route selected from the group consisting of oral, intranasal, inhalation, transdermal, topical, rectal, vaginal, buccal, injection, sublingual, or combination thereof.

16. The method and compound of claims 1, 2, 3, 4, 5, 6, 7, 8, 9, 10 and 11, where said compound is administered in a dosage form selected from the group consisting of a tablet, a liquid dosage form, a hard gelatin capsule, a soft gelatin capsule, an HPMC capsule, an inhalant, an injectable, a transdermal, a buccal, a sublingual, and a rectal or a vaginal suppository, a skin cleanser, a skin toner, a skin moisturizer, a topical skin mask, a topical skin composition.

17. The method and compound of claims 1, 2, 3, 4, 5, 6, 7, 8, 9, 10 and 11, where said compound is administered in combination and/or includes one or more other medications, dietary supplements or food products.

18. The method and compound of claims 1, 2, 3, 4, 5, 6, 7, 8, 9, 10 and 11, where said compound is used for treating of at least one of upper respiratory tract infection (common cold) or an acute respiratory illness (flu) of viral or bacterial origin, acid refluxing, phytosis and mycosis, gastric and duodenal ulcers, Huntington's Disease; Wilson's Disease; Parkinson's Disease; chronic cough; cough associated with Asthma, allergic reaction, respiratory disease, gastro-oesophageal reflux disease (GORD) and post nasal drip syndrome (PNDS); obesity and body weight reduction; diabetes; metabolic and endocrine diseases and disorders; autoimmune system responses (allergic reactions), athetosis-related to damage or degeneration of basal ganglia; gastritis; acid overproduction; acid reflux; peptic ulcers; minor tranquilizers, alcohol, cocaine, (meta)amphetamine, and opioid withdrawal syndromes; symptoms or side effects associated with anti-retroviral therapy, chemotherapy and radiation therapy; AIDS; rheumatoid arthritis; osteoarthritis; fibromyalgia; pain and spasticity symptoms associated with Multiple Sclerosis, Neuromuscular Junction Disorder, autoimmune diseases and disorders, motor neuron diseases and disorders, neurodegenerative diseases and disorders; pain associated with cancer; trauma; athletic performance; migraine; surgical intervention or medical treatment; stroke; heart attack; dental and gum pain; abdominal pain; bone pain, muscle pain; neurological pain; stomach ulcers-related pain; gallbladder disease-related pain; Central Pain Syndrome (CPS); sports trauma; chronic pain disorder (nociceptive pain, neuropathic pain, chronic back or leg pain, painful neuropathies, Complex Regional Pain Syndrome), and acute pain.

19. The method and compound of claims 1, 3, 6, 7, 9 and 11, where said akuammine, dihydroakuammine, akuammidine, pseudoakuammine, akuammicine, akuammigine, pseudoakuammigine, akuammiline and akuammenine, picraphylline, picracine, picraline, picralicine, picratidine, picranitine, burnamine, pericalline and pericineby, reserpinine, majoridine, vincamajoridine, vincamine, vincamajine, perivincine, pubescine, vinine, vincawajine, majorinine, 10-methoxyvinorineor, indole monoterpene-type alkaloids, pyrrolidinoindoline-type alkaloids, mitragynine, pseudoindoxyl, 7-hydroxymitragynine alkaloids are one or more of (i) natural substance that has been purified or modified; (ii) synthetically derived substance, not extracted from a plant; (iii) semi-synthetic substance; (iv) esterified substance; (v) active metabolites of any of the foregoing, (vi) pro-drugs of any of the foregoing; (vii) analogs of any of the foregoing; (viii) derivatives of any of the foregoing; (ix) and mixtures thereof.

20. The method and compound of claims 1, 2, 3, 4, 5, 6, 7, 8, 9 and 10, where said extract of Picralima nitida plant is the extract of Vinca major plant.

21. The method and compound of claims 1, 2, 3, 4, 5, 6, 7, 8, 9 and 10, where said extract is obtained from any part of the plant by distillation, or solvent extraction, or maceration, or enfleurage, or cold-press extraction, or fractioning, or a combination thereof.

22. The method and compound of claims 1, 2, 3, 4, 5, 6, 7, 8, 9, 10 and 11, where said compound is dispersed in an inert water-soluble carrier at solid (solid dispersion) or liquid state and/or incorporated into a lipid carrier.

23. The method and compound of claims 1, 2, 3, 4, 5, 6, 7, 8, 9, 10 and 11, where said compound is an ingestible preparation, where the pharmacologically inactive substance is a food product or a food additive.
